(19) United States
(12) Patent Application Publication
(10) Pub. No.: US 2024/0299615 A1
(43) Pub. Date: Sep. 12, 2024

(54) HYDROGEL INCLUDING PHENOL DERIVATIVE-MODIFIED CELLULOSE AND USE THEREOF

(71) Applicant: INDUSTRY-ACADEMIC COOPERATION FOUNDATION, YONSEI UNIVERSITY, Seoul (KR)

(72) Inventors: Seung Woo CHO, Seoul (KR); Yi Sun CHOI, Seoul (KR); Ji Hoon JEON, Seoul (KR)

(21) Appl. No.: 18/702,663

(22) PCT Filed: Apr. 11, 2022
(86) PCT No.: PCT/KR2022/005204

§ 371 Date: Apr. 18, 2024

(30) Foreign Application Priority Data
Apr. 9, 2021 (KR) 10-2021-0046484
Apr. 8, 2022 (KR) 10-2022-0044173

(51) Int. Cl. A61L 24/00; A61K 9/06; A61K 45/06; A61K 47/18; A61K 47/38; A61L 24/08; A61L 27/20; A61L 27/38; A61L 27/52; C08B 15/06; C08B 15/10; C08J 3/075; C08J 3/24; C08L 1/08

(52) U.S. Cl. CPC A61L 24/0031; A61K 9/06; A61K 45/06; A61K 47/18; A61K 47/38; A61L 24/08; A61L 27/20; A61L 27/3834; A61L 27/52; C08B 15/06; C08B 15/10; C08J 3/075; C08J 3/24; C08L 1/08; A61L 2400/04; C08J 2301/08; C08L 2203/02; C08L 2312/00

(57) ABSTRACT

A hydrogel including phenol derivative-modified cellulose, a use thereof, and a preparation method therefor are described. Physical properties of the hydrogel can be modulated by adjusting concentrations of and oxidation conditions for the phenol derivative-modified cellulose. In addition, the hydrogel of the present invention can find applications in various fields due to its properties, such as hemostasis, blood coagulation acceleration, tissue adhesion, cell culture, cell transplantation, drug delivery, etc.

### TECHNICAL FIELD

The present invention relates to a hydrogel comprising cellulose modified with a phenol derivative, the use thereof, and a production method therefor.

### BACKGROUND ART

The need for functional medical materials in surgical operations and treatments is increasing, and various types of products have already been developed and used clinically.

Thereamong, most of currently commercially available hemostatic agents are composed of fibrin-based derivatives, and act in the way of simple clotting that mix and react a fibrinogen protein solution and a thrombin protein solution to form fibrin clots, which physically plug the bleeding site. In this case, a large amount of the hemostatic agent is required to obtain a sufficient hemostatic effect, and the resulting fibrin clots have the potential to cause side effects that slow skin regeneration or cause adhesion to surrounding tissues.

To overcome the problems of conventional hemostatic agents that physically induce hemostasis as described above, there is an increasing need for a new technology that can improve the hemostatic performance of hemostatic agents and solve side effects caused by excessive fibrin clots. Further, there is an increasing need for functional medical materials capable of performing the above-described complex functions with a single product.

### DISCLOSURE

### Technical Problem

An object of the present invention is to provide a hydrogel comprising cellulose modified with a phenol derivative.

Another object of the present invention is to provide a hemostatic agent composition comprising the hydrogel.

Still another object of the present invention is to provide a tissue adhesive composition comprising the hydrogel.

Yet another object of the present invention is to provide a composition for cell culture and transplantation comprising the hydrogel.

Still yet another object of the present invention is to provide a composition for drug delivery comprising the hydrogel.

A further object of the present invention is to provide a method for producing a hydrogel comprising cellulose modified with a phenol derivative, the method comprising steps of: a) substituting the hydroxyl (—OH) group of carboxymethyl cellulose with a phenol derivative; and b) crosslinking the substituted carboxymethyl cellulose to form a hydrogel.

### Technical Solution

One aspect of the present invention provides a hydrogel comprising cellulose modified with a phenol derivative.

In one embodiment of the present invention, the phenol derivative may be dopamine

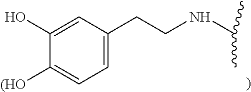

or 5-hydroxydopamine

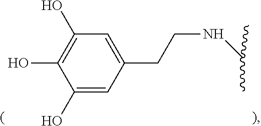 [SMILES: *NCCc1cc(O)c(O)c(O)c1]

and the cellulose may be any one or more of carboxymethyl cellulose, hydroxypropyl methylcellulose, cellulose acetate phthalate, hydroxypropyl methylcellulose acetate/succinate, hydroxyethyl cellulose, ethyl methyl cellulose, hydroxypropyl cellulose, cellulose propionate, and cellulose acetate butyrate.

In one embodiment of the present invention, the hydrogel may comprise a structure represented by Formula 1 below:

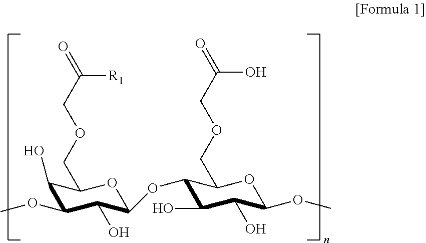

wherein
R₁ may be

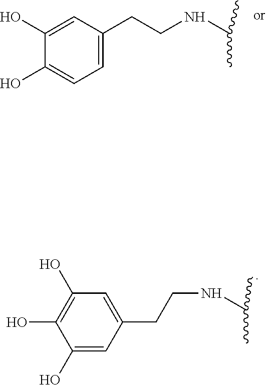 [SMILES: *NCCc1ccc(O)c(O)c1 ; *NCCc1cc(O)c(O)c(O)c1]

In one embodiment of the present invention, the phenol derivative and the cellulose may be at a molar ratio of 1:3 to 3:1.

In one embodiment of the present invention, the hydrogel may be used for one or more purposes selected from the group consisting of blood clotting promotion, hemostasis, cell differentiation promotion, cell culture, cell transplantation, and drug delivery.

In one embodiment of the present invention, the hydrogel may be adhesive, and the hydrogel may be biodegradable.

Another aspect of the present invention provides a hemostatic agent composition comprising the hydrogel, wherein the hemostatic agent may be in the form of an adhesive patch or film.

Still another aspect of the present invention provides a tissue adhesive composition comprising the hydrogel.

Yet another aspect of the present invention provides a composition for cell culture and transplantation comprising the hydrogel.

Still yet another aspect of the present invention provides a composition for drug delivery comprising the hydrogel.

In one embodiment of the present invention, the drug may be loaded in the hydrogel.

In one embodiment of the present invention, the drug may comprise one selected from the group consisting of immune cell activators, anticancer agents, therapeutic antibodies, antibiotics, antibacterial agents, antiviral agents, anti-inflammatory agents, contrast agents, protein drugs, growth factors, cytokines, peptide drugs, hair growth agents, anesthetics, and combinations thereof.

A further aspect of the present invention provides a method for producing a hydrogel comprising cellulose modified with a phenol derivative, the method comprising steps of: a) substituting cellulose with a phenol derivative; and b) crosslinking the substituted cellulose to form a hydrogel.

In one embodiment of the present invention, the step of substituting cellulose with a phenol derivative may comprise substituting the hydroxyl (—OH) group of cellulose with R₁ to produce a compound represented by Formula 1 below:

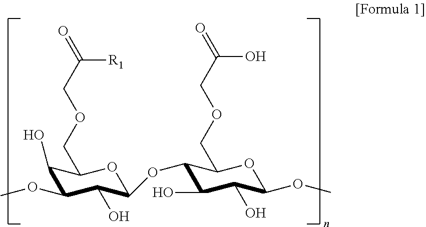

wherein
R₁ may be

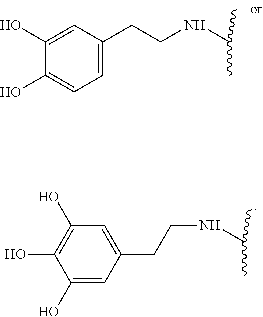 [SMILES: *NCCc1ccc(O)c(O)c1 ; *NCCc1cc(O)c(O)c(O)c1]

In one embodiment of the present invention, step b) of crosslinking the substituted cellulose to form a hydrogel may comprise treating the substituted cellulose with any one or more of NaOH, NaIO₄, Na₂S₂O₈, Fe³⁺, HNO₃, MnO₄²⁻, and H₂SO₄.

### Advantageous Effects

The physical properties of the hydrogel comprising cellulose modified with a phenol derivative according to the present invention may be adjusted by adjusting the degree of modification with a phenol group, the concentration of cellulose, and oxidation conditions.

In addition, the hydrogel of the present invention exhibits various effects such as hemostasis, blood clotting promotion, cell transplantation, drug delivery, and cell differentiation promotion, and thus may be used for single or multiple purposes. Furthermore, the hydrogel of the present invention has little cytotoxicity, is biodegradable, and has excellent biocompatibility, and thus it has a wide range of applications.

### BRIEF DESCRIPTION OF DRAWINGS

FIGS. 1A-1C show a process for synthesis of cellulose modified with a phenol derivative (catechol group) (FIG. 1A), the results of 1H-NMR analysis of cellulose modified with a phenol derivative (catechol group) (FIG. 1B), and the results of analyzing the degree of substitution (FIG. 1C).

FIGS. 2A-2C show a process for crosslinking of cellulose modified with a phenol derivative (catechol group) (FIG. 2A), and the results of UV-Vis analysis (FIG. 2B) and FT-IR analysis (FIG. 2C) that indicate the structure of crosslinked cellulose modified with a phenol derivative (catechol group).

FIGS. 3A-3C show the crosslinking of a phenol derivative (catechol group)-modified cellulose hydrogel by oxidation (FIG. 3A), and the results of measuring the crosslinking rate (solution-gel transition time and the time taken for completion of hydrogel formation) of phenol derivative (catechol group)-modified cellulose depending on concentration (FIG. 3B) and pH (FIG. 3C) conditions.

FIGS. 4A-4D show the results of measuring the elastic moduli of hydrogels produced using different concentrations of phenol derivative (catechol group)-modified cellulose (FIGS. 4A to 4C), and the results of analyzing the swelling profiles of the hydrogels (FIG. 4D).

FIGS. 5A-5C show the results of analyzing the adhesion forces of hydrogels, produced using different concentrations of phenol derivative (catechol group)-modified cellulose, to metal plates (FIG. 5A), and the results of analyzing the adhesion forces of the hydrogels to liver tissue (FIGS. 5B and 5C).

FIGS. 6A-6E show the results of analyzing the adhesion forces of hydrogels, produced using different concentrations of phenol derivative (catechol group)-modified cellulose, to intestinal tissue.

FIG. 7 depicts photographs showing the internal structures of hydrogels produced using different concentrations of phenol derivative (catechol group)-modified cellulose.

FIGS. 8A-8C show the results of evaluating biocompatibility through 3D cell culture. Specifically, FIG. 8A shows the results of live/dead staining of human adipose-derived stem cells cultured in a phenol derivative (catechol group)-modified cellulose hydrogel, FIG. 8B is the cell viability results obtained by quantifying the results shown in FIG. 8A, and FIG. 8C shows the results of measuring the amount of TNF-α secretion from macrophages co-cultured with a phenol derivative (catechol group)-modified cellulose hydrogel.

FIGS. 9A-9B show the results of analyzing in vivo toxicity. Specifically, FIG. 9A shows the results of hematoxylin & eosin (H&E) staining of a tissue area implanted with a phenol derivative (catechol group)-modified cellulose hydrogel, and FIG. 9B shows the results of toluidine blue staining of the tissue area.

FIGS. 10A-10C show the results of preparing a hemostatic agent composition using the hydrogel of the present invention and evaluating the hemostatic performance of the composition in a bleeding model. Specifically, FIGS. 10A and 10B depict photographs showing the results of experiments conducted to evaluate the hemostatic performance, and graphs showing the results of quantifying the hemostatic performance, and FIG. 10C shows the results of H&E histological analysis.

FIGS. 11A-11D show the results of producing the hydrogel of the present invention in the form of a patch and evaluating the usefulness thereof. Specifically, FIG. 11A depicts photographs showing the appearance of the patch before and after crosslinking (oxidation) of phenol derivative-modified cellulose, and FIGS. 11B to 11D shows the results of measuring the physical properties of the patch.

FIGS. 12A-12C show a process for synthesis of cellulose modified with a phenol derivative (gallol group) (FIG. 12A), and depicts graphs showing the results of 1H-NMR analysis of the cellulose (FIG. 12B) and the results of analyzing the degree of substitution of the cellulose (FIG. 12C).

FIGS. 13A-13D show a process of crosslinking phenol derivative (gallol group)-modified cellulose (FIG. 13A), and the results of UV-Vis analysis (FIGS. 13B and 13C) and FT-IR analysis (FIG. 13D) of the cellulose.

FIGS. 14A-14D show the results of measuring the elastic moduli of hydrogels produced using different concentrations of phenol derivative (gallol group)-modified cellulose and different oxidizing agents.

FIGS. 15A-15B show the results of analyzing the swelling profiles (FIG. 15A) and internal structures of hydrogels produced using different concentrations of phenol derivative (gallol group)-modified cellulose and different oxidizing agents.

FIGS. 16A-16B show the results of analyzing the adhesion forces of hydrogels produced using different concentrations of phenol derivative (gallol group)-modified cellulose.

FIGS. 17A-17B and 18A-18B show the results of evaluating the pH sensitivity of a hydrogel. Specifically, FIGS. 17A and 17B show pH-dependent changes in the size of a hydrogel, and FIGS. 18A and 18B show the pH-dependent internal structure and pore size of a hydrogel.

FIGS. 19A-19B show the results of evaluating the applicability of a hydrogel as a pH-sensitive drug delivery system. Specifically, it shows the amount of pH-dependent BSA release from the hydrogel.

FIGS. 20A-20B show the results of analyzing the biocompatibility of hydrogels. Specifically, it shows the results of live/dead staining of human adipose-derived stem cells three-dimensionally cultured in phenol derivative (gallol group)-modified cellulose hydrogels (FIG. 20A), and graphs showing the cell viabilities obtained by quantifying the results (FIG. 20B).

FIGS. 21A-21C and 22 show the applicability of the hydrogel of the present invention. Specifically, FIGS. 21A-21C show the results of evaluating the applicability of the hydrogel as a filler material, and FIG. 22 shows the results of evaluating the applicability of the hydrogel as a material for cell transplantation.

FIGS. 23A-23D and 24A-24B show the applicability of the hydrogel (CMC-PG) of the present invention. Specifically, FIGS. 23A-23D show a produced patch and the results of analyzing the properties of the patch, and FIGS. 24A-24B show the results of evaluating the applicability of the hydrogel as a hemostatic agent.

### BEST MODE

One aspect of the present invention provides a hydrogel comprising cellulose modified with a phenol derivative.

In one embodiment of the present invention, the phenol derivative may be dopamine

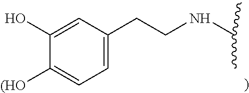

or 5-hydroxydopamine

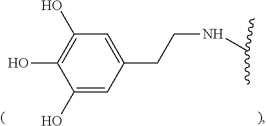 [SMILES: *NCCc1cc(O)c(O)c(O)c1]

and
the cellulose may be any one or more of carboxymethyl cellulose, hydroxypropyl methylcellulose, cellulose acetate phthalate, hydroxypropyl methylcellulose acetate/succinate, hydroxyethyl cellulose, ethyl methyl cellulose, hydroxypropyl cellulose, cellulose propionate, and cellulose acetate butyrate.

In the present invention, “dopamine” is a catecholamine-based organic compound, which is a type of neurotransmitter that is released for signaling between brain neurons. Since dopamine is a catecholamine compound made from tyrosine, it is converted into norepinephrine and epinephrine through a biochemical process, and plays an important role not only in the central nervous system, but also in the kidneys, hormones, and cardiovascular system. Thus, dopamine has been studied extensively. In addition, dopamine is oxidized to form a quinone structure, which has excellent adhesive properties, and polydopamine formed by polymerization of the quinone also serves as a bridge.

In the present invention, “carboxymethyl cellulose (CMC)” is a type of oxidized cellulose, which is a cellulose derivative with proven safety and efficacy, and the use thereof is easy to approve. In addition, it is a material that is naturally degradable, is harmless to the human body, and can replace existing absorbent chemical products. Carboxymethyl cellulose (CMC) is a material obtained by reacting wood-based cellulose with NaOH alkaline treatment, followed by reaction with monochloroacetic acid (MCA), and has already been widely used as a viscosity regulator, an additive, etc. in the food industry.

In the present invention, “hydrogel” is a gel containing water as a dispersion medium or water as a basic component. The hydrogel in the present invention is characterized by comprising cellulose modified with a phenol derivative.

In addition, the physical properties of hydrogels, such as elasticity and adhesion, are very important factors for hydrogels that are used as scaffolds for the treatment of defects in certain tissues, such as articular cartilage or bone, which are exposed to higher loads. The physical properties of the hydrogel of the present invention may be adjusted by adjusting oxidation conditions, and thus the hydrogel may be produced to satisfy conditions and used.

In one embodiment of the present invention, the hydrogel may comprise a structure represented by Formula 1 below:

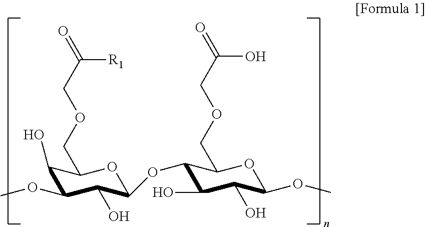

wherein R₁ may be

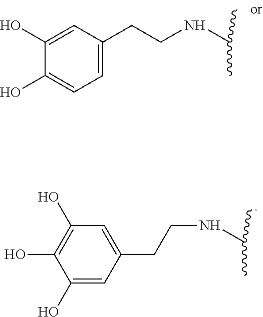 [SMILES: *NCCc1ccc(O)c(O)c1 ; *NCCc1cc(O)c(O)c(O)c1]

In one embodiment of the present invention, the hydrogel may be used for one or more purposes selected from the group consisting of blood clotting promotion, hemostasis, cell differentiation promotion, cell culture, cell transplantation, and drug delivery. The hydrogel may be adhesive, and the hydrogel may be biodegradable.

In one embodiment of the present invention, the phenol derivative and the cellulose may be at a molar ratio of 1:3 to 3:1, specifically 1:2 to 1:1, more specifically 1:2.

Another aspect of the present invention provides a hemostatic agent composition comprising the hydrogel. Specifically, the hemostatic agent is able to stop bleeding and induce blood clot formation.

In one embodiment of the present invention, the hemostatic agent may be in the form of an adhesive patch or film. In one example of the present invention, it was confirmed that, when the hemostatic agent was applied to an animal model in an adhesive form, it exhibited an excellent hemostatic effect, suggesting that it is applicable in a form that can adhere to a surface. The surface may be the surface of any tissue of the body and is not limited to a specific site.

Still another aspect of the present invention provides a tissue adhesive composition comprising the hydrogel. Since the hydrogel exhibits adhesive properties as described above, it may be used for adhesion between tissues.

Yet another aspect of the present invention provides a composition for cell culture and transplantation comprising the hydrogel. Specifically, the culture may be three-dimensional culture, and the cells to be cultured and transplanted may be stem cells, hematopoietic stem cells, hepatocytes, fibrocytes, epithelial cells, mesothelial cells, endothelial cells, muscle cells, nerve cells, immune cells, adipocytes, chondrocytes, osteocytes, blood cells, or skin cells, without being limited thereto. The hydrogel of the present invention may be applied to any type of cells capable of growing on the hydrogel. More specifically, the culture may be co-culture of two or more cell types.

Still yet another object of the present invention is to provide a composition for drug delivery comprising the hydrogel.

In one embodiment of the present invention, the drug may be loaded in the hydrogel.

In one embodiment of the present invention, the drug may comprise one selected from the group consisting of immune cell activators, anticancer agents, therapeutic antibodies, antibiotics, antibacterial agents, antiviral agents, anti-inflammatory agents, contrast agents, protein drugs, growth factors, cytokines, peptide drugs, hair growth agents, anesthetics, and combinations thereof.

A further aspect of the present invention provides a method for producing a hydrogel comprising cellulose modified with a phenol derivative, the method comprising steps of: a) substituting cellulose with a phenol derivative; and b) crosslinking the substituted cellulose to form a hydrogel.

As described above, the cellulose may be any one or more of carboxymethyl cellulose, hydroxypropyl methylcellulose, cellulose acetate phthalate, hydroxypropyl methylcellulose acetate/succinate, hydroxyethyl cellulose, ethyl methyl cellulose, hydroxypropyl cellulose, cellulose propionate, and cellulose acetate butyrate, and the phenol derivative may be dopamine

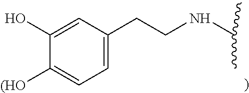

or 5-hydroxydopamine

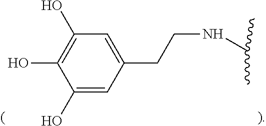 [SMILES: *NCCc1cc(O)c(O)c(O)c1]

In one embodiment of the present invention, the step of substituting cellulose with a phenol derivative may comprise substituting the hydroxyl (—OH) group of cellulose with R₁ to produce a compound represented by Formula 1 below:

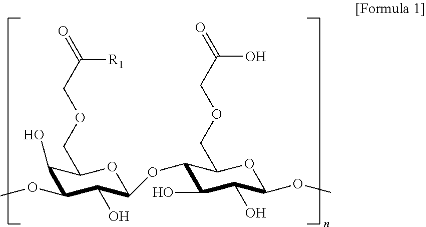

wherein
R₁ is

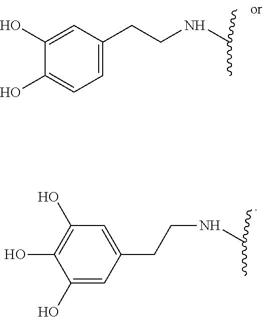 [SMILES: *NCCc1ccc(O)c(O)c1 ; *NCCc1cc(O)c(O)c(O)c1]

Specifically, this step comprises substituting the hydroxyl group located at the R₁ position in carboxymethyl cellulose with the phenol derivative of R₁. The substitution may be performed by mixing carboxymethyl cellulose and the phenol derivative of R₁, and then treating the mixture with EDC/NHS at room temperature for 24 hours. Specifically, the substitution may be performed by mixing carboxymethyl cellulose with EDC/NHS, stirring the mixture at pH 4 to 8, more specifically pH 5.0 to 6.0, adding the phenol derivative thereto, and then adjusting the pH to 3.8 to 6.0, specifically pH 4.5 to 5.0 when the phenol derivative is dopamine, or 3.8 to 5.0 when the phenol derivative is 5-hydroxydopamine, followed by stirring.

In one embodiment of the present invention, step b) of crosslinking the substituted cellulose to form a hydrogel may comprise treating the substituted cellulose with any one or more of NaOH, NaIO₄, Na₂S₂O₈, Fe³⁺, HNO₃, MnO₄²⁻, and H₂SO₄.

Specifically, crosslinking between the phenol derivatives of the substituted carboxymethyl cellulose may be performed, and as a result of crosslinking between the phenol derivatives, quinone, a semi-quinone intermediate, phenoxy radicals, etc. may be formed.

### MODE FOR INVENTION

Hereinafter, one or more embodiments will be described in more detail by way of examples. However, these examples are intended to illustrate one or more embodiments and the scope of the present invention is not limited to these examples.

### Example 1: Synthesis of Dopamine-Modified Carboxymethyl Cellulose Derivative and Hydrogel

### Example 1-1: Synthesis of Dopamine-Modified Carboxymethyl Cellulose

### Derivative

To synthesize a dopamine-modified carboxymethyl cellulose derivative, carboxymethyl cellulose (CMC) was modified with dopamine using EDC/NHS chemical reaction.

Specifically, carboxymethyl cellulose was dissolved in triple-distilled water (TDW) or 2-(N-morpholino) ethanesulfonic acid (MES) buffer solution at pH 5.5. 1-(3-dimethylaminopropyl)-3-ethylcarbodiimide hydrochloride (EDC, Thermo Fisher Scientific, Waltham, MA, USA) and N-hydroxysuccinimide (NHS, Sigma-Aldrich, St. Louis, MO, USA) were added to the solution, followed by stirring at pH 5.5 to 6.0 for 30 minutes.

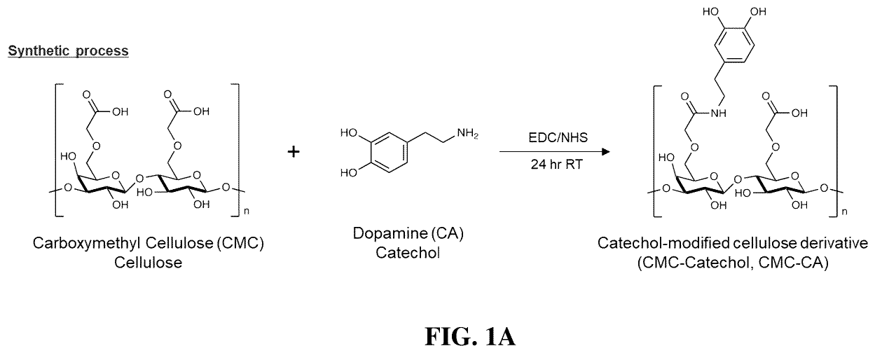

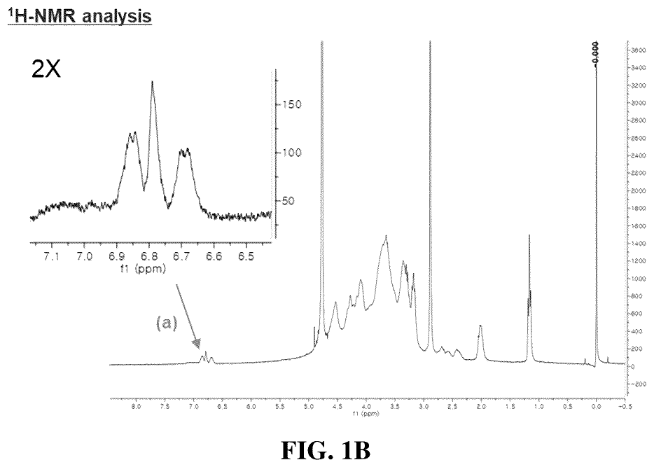

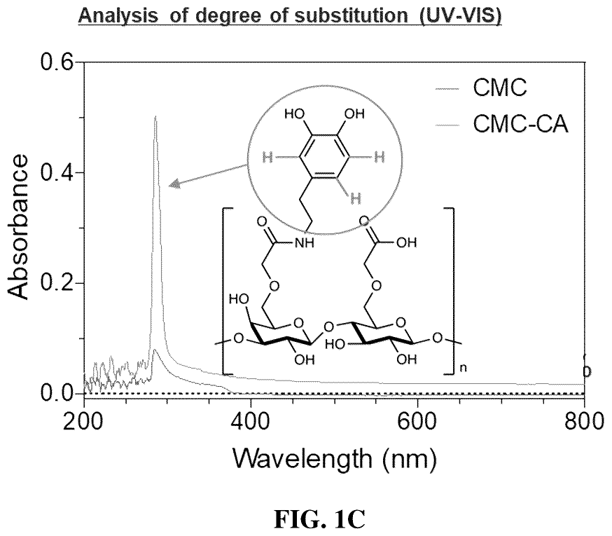

Dopamine was added to the solution which was then stirred overnight at room temperature and pH 5.5 to 6.0, thereby synthesizing a dopamine-modified carboxymethyl cellulose derivative. At this time, carboxymethyl cellulose (CMC):EDC:NHS:dopamine (CA) were mixed and reacted at a molar ratio of 1:2:2:2 (FIG. 1A).

The dopamine-modified carboxymethyl cellulose derivative produced through the above-described process was analyzed by 1H-NMR (300 MHZ, Bruker, Billerica, MA, USA). As a result, as shown in FIG. 1B, it was confirmed that dopamine was bound to carboxymethyl cellulose, and the peak values corresponding to dopamine and carboxymethyl cellulose, respectively, appeared.

In addition, the degree of substitution with the catechol group for the existing CMC polymer was analyzed by UV-VIS spectrometry at a wavelength of 280 nm. As a result, as shown in FIG. 1C, it could be seen that about 4% of the carboxyl groups were substituted with the catechol group.

### Example 1-2. Production of Hydrogel

A cellulose hydrogel was produced by crosslinking the dopamine-modified carboxymethyl cellulose derivative of Example 1-1. Specifically, a hydrogel was produced by inducing crosslinking by an oxidation reaction with sodium periodate (NaIO₄).

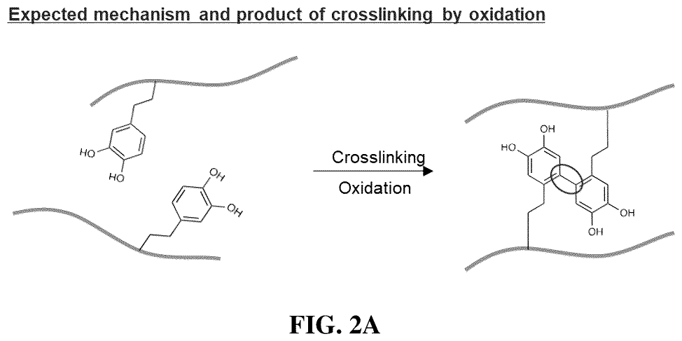

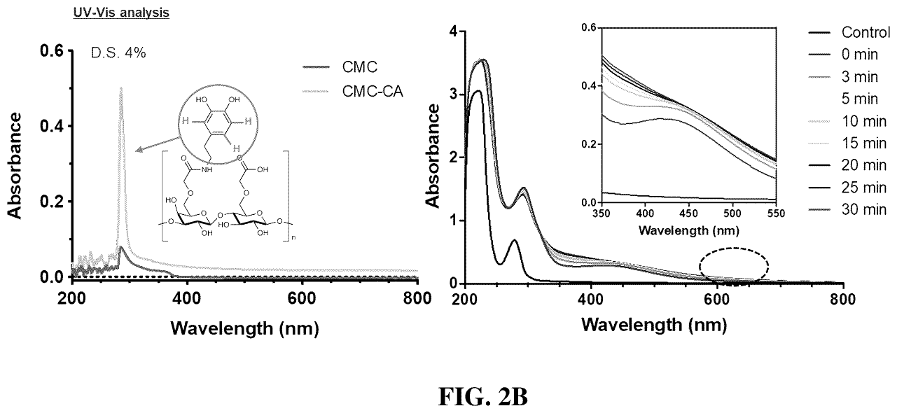

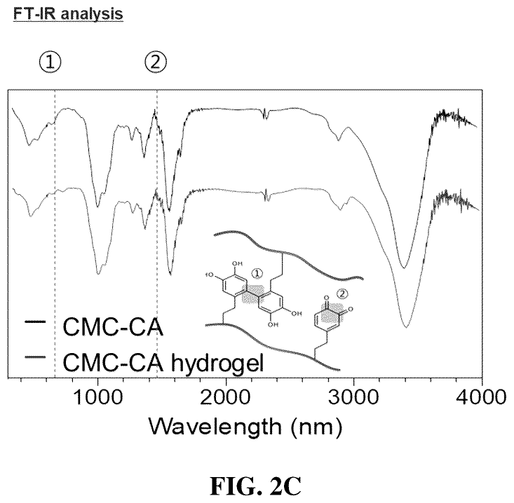

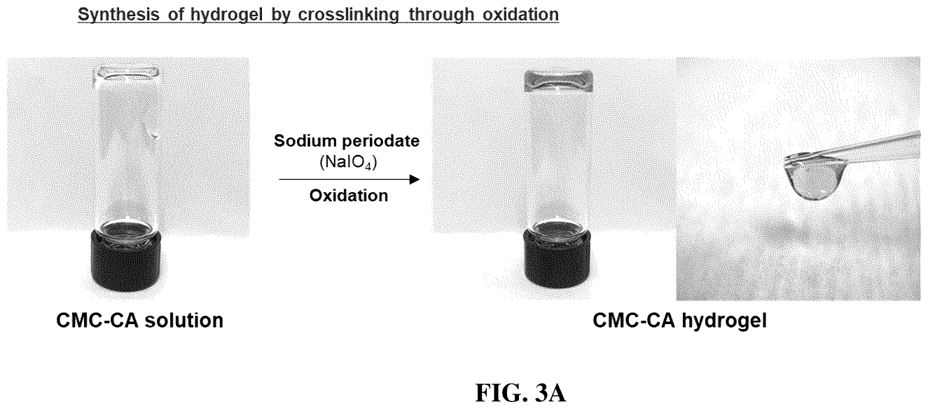

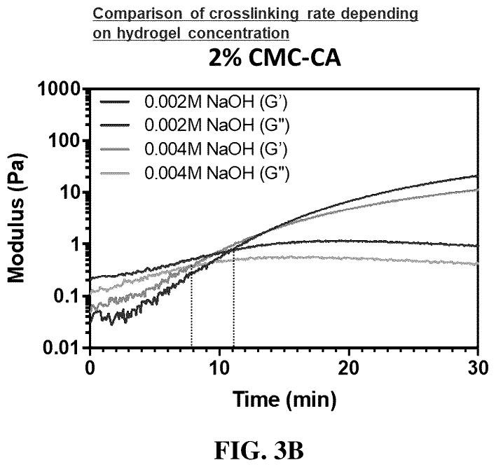

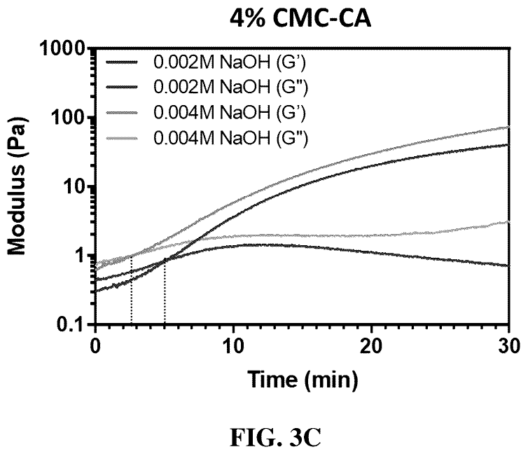

As shown in FIG. 2A, it was confirmed that crosslinking of the carboxymethyl cellulose derivative was achieved by chemical bonding between the oxidized catechol groups, and the obtained hydrogel was slightly yellow (see FIG. 3A).

In order to specifically confirm the crosslinking of the carboxymethyl cellulose hydrogel, the spectra before and after oxidation were compared through ultraviolet-visible 9 UV-Vis) spectroscopy.

As a result of UV-Vis analysis, the presence of a catechol peak (˜280 nm) before oxidation was confirmed, and as a result of performing analysis depending on the reaction time after oxidation, it was confirmed that a peak (˜400 nm) corresponding to quinone and a peak (˜460 nm) corresponding to a material produced by an additional reaction between quinone and catechol increased (FIG. 2B).

In addition, the chemical mechanism of the crosslinking reaction was examined through FT-IR analysis. Specifically, it was confirmed that, when the CMC-CA polymer was treated with an oxidizing agent (NaIO₄), an oxidized catechol structure was formed (\textcircled{2} ˜1,400 m), and crosslinking of the polymer proceeded while a catechol dimer was produced through a reaction between oxidized catechols (\textcircled{1} ˜700 m) (FIG. 2C).

In addition, it was confirmed that the dopamine-modified carboxymethyl cellulose hydrogel was produced by the process of crosslinking by oxidation as described above (FIG. 3A).

### Experimental Example 1: Characterization of Dopamine-Modified Carboxymethyl Cellulose

### Experimental Example 1-1. Comparison of Crosslinking Rate Depending on Hydrogel Derivative Concentration

In the hydrogel production step of Example 1-2, dopamine-modified carboxymethyl cellulose solutions were prepared at concentrations of 2 wt % and 4 wt %, and the sol-gel transition time of the dopamine-modified carboxymethyl cellulose derivative was measured under varying pH conditions (sodium hydroxide molar concentrations).

As a result, it was confirmed that the formation rate of the dopamine-modified carboxymethyl cellulose hydrogel increased as the concentration of the dopamine-modified carboxymethyl cellulose and the concentration of sodium hydroxide increased (FIG. 3B).

### Experimental Example 1-2. Examination of Changes in Physical Properties Depending on Hydrogel Derivative Concentration

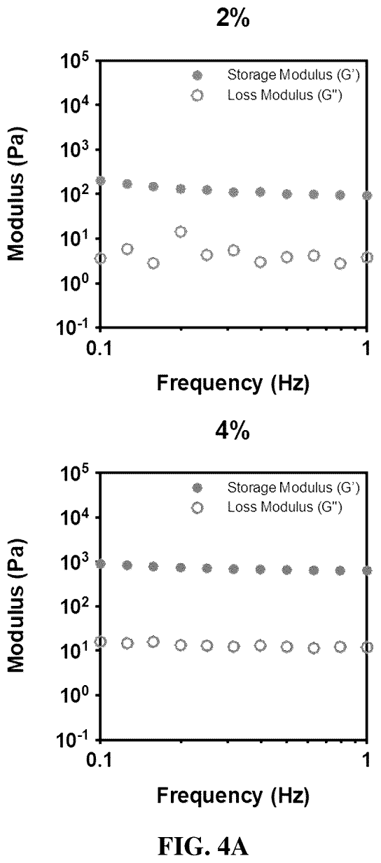

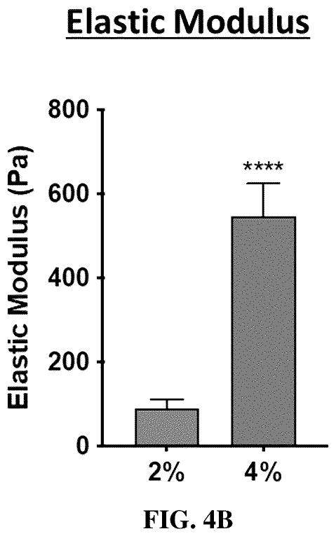

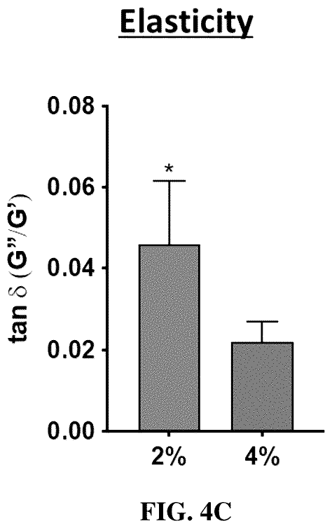

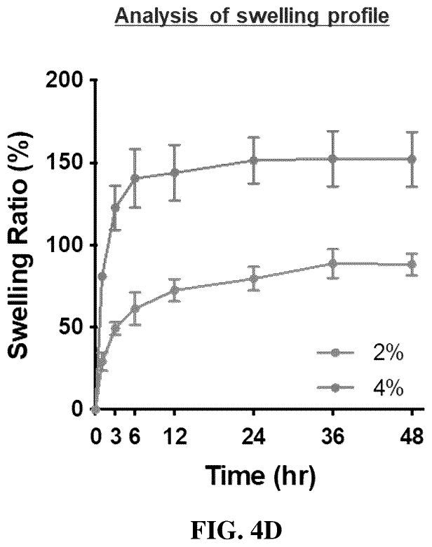

The viscoelastic moduli of the hydrogels produced using dopamine-modified carboxymethyl cellulose solutions prepared at concentrations of 2 wt % and 4 wt % were analyzed by measuring the storage modulus (G′) and loss modulus (G″) using an MCR 102 rheometer (Anton Paar, Ashland, VA, USA) in frequency sweep mode in the frequency range of 0.1 to 1 Hz (FIG. 4A). The elasticity of the hydrogel was expressed by calculating the average storage modulus at 1 Hz (n=3).

As shown in FIGS. 4B and 4C, it was confirmed that the mechanical property (elastic modulus) and elasticity of the hydrogel increased as the concentration of the dopamine-modified carboxymethyl cellulose increased. From these results, it was confirmed that hydrogels having various physical properties can be formed by adjusting the concentration of the dopamine-modified carboxymethyl cellulose.

### Experimental Example 1-3. Analysis of Swelling Profile Depending on Hydrogel Derivative Concentration

The swelling profile of the hydrogel depending on the concentration of dopamine-modified carboxymethyl cellulose was analyzed.

Specifically, the present inventors compared the swelling profiles of hydrogels produced by preparing dopamine-modified carboxymethyl cellulose solutions at concentrations of 2 wt % and 4 wt % and treating the solutions with 4.5 mg/ml of sodium periodate (NaIO₄) and 0.004 M of sodium hydroxide (NaOH).

As a result, as shown in FIG. 4D, it was confirmed that the hydrogel produced using the 4 wt % dopamine-modified carboxymethyl cellulose solution swelled more than the hydrogel produced using the 2 wt % dopamine-modified carboxymethyl cellulose solution, suggesting that the swelling profile of the hydrogel is adjustable depending on the concentration of the dopamine-modified carboxymethyl cellulose.

### Experimental Example 1-4. Analysis of Adhesive Properties of Hydrogels

To evaluate the adhesive properties of the hydrogels produced in the present invention, rheological analysis was performed.

Specifically, hydrogels produced by preparing dopamine-modified carboxymethyl cellulose solutions at concentrations of 2 wt % and 4 wt % and treating the solutions with 4.5 mg/ml of sodium periodate and 0.004 M of sodium hydroxide (NaOH) were attached to metal plates, and then the adhesion forces thereof were measured and expressed numerically.

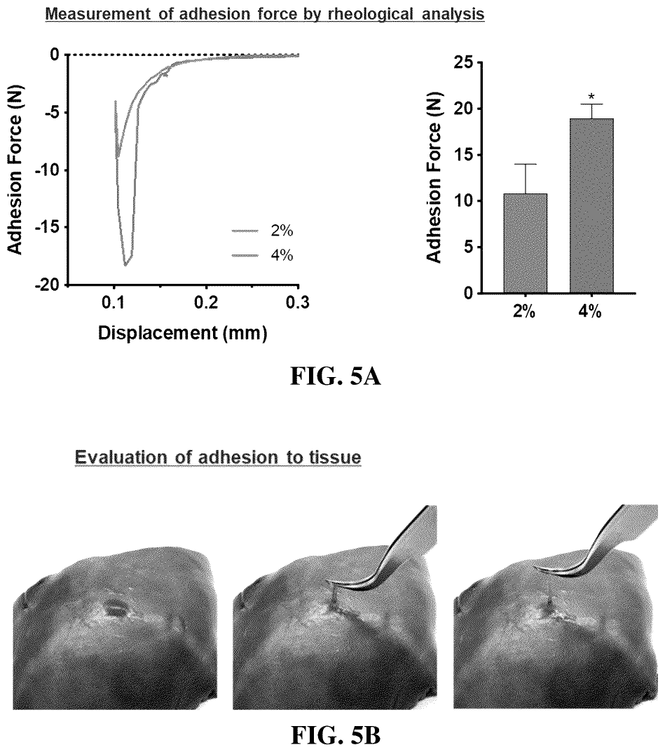

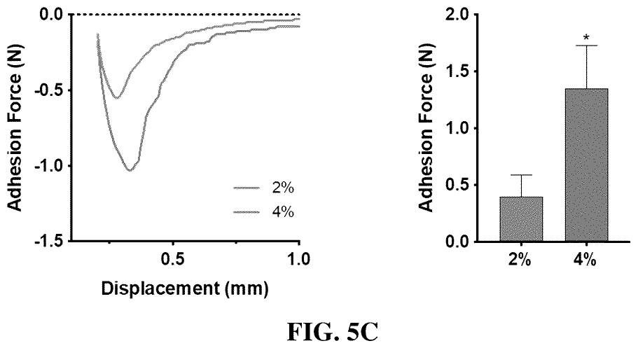

As a result, as seen in FIG. 5A, it was confirmed that the adhesion force of the hydrogel increased as the concentration of the dopamine-modified carboxymethyl cellulose increased, suggesting that the adhesion force is adjustable by adjusting the concentration of the dopamine-modified carboxymethyl cellulose.

### Experimental Example 1-5. Evaluation of Adhesion of Hydrogel to In Vivo Tissues (Liver and Intestine)

It was examined whether the dopamine-modified carboxymethyl cellulose hydrogel of the present invention would have sufficient adhesion forces even in in vivo tissues.

Specifically, after applying the hydrogel to liver tissue, the applied hydrogel was pulled, and it was measured whether the hydrogel remained adhered well to the tissue. In addition, the adhesion force of the hydrogel applied to the tissue was measured using a rheological analysis device.

As a result, as seen in FIG. 5B, it was confirmed that the hydrogel remained adhered well to the liver tissue while showing high adhesiveness. In addition, as shown in FIG. 5C, the results of rheological analysis showed that the hydrogel produced using the dopamine-modified carboxymethyl cellulose solution prepared at a concentration of 4 wt % had a high adhesion force to the tissue, suggesting that the hydrogel has a sufficient adhesion force to adhere to and remain in in vivo tissue.

In addition, after applying the hydrogel to intestinal tissue, the applied hydrogel was pulled, and it was checked whether the hydrogel remained adhered well to the tissue. Further, the adhesion force of the hydrogel applied to the tissue was measured.

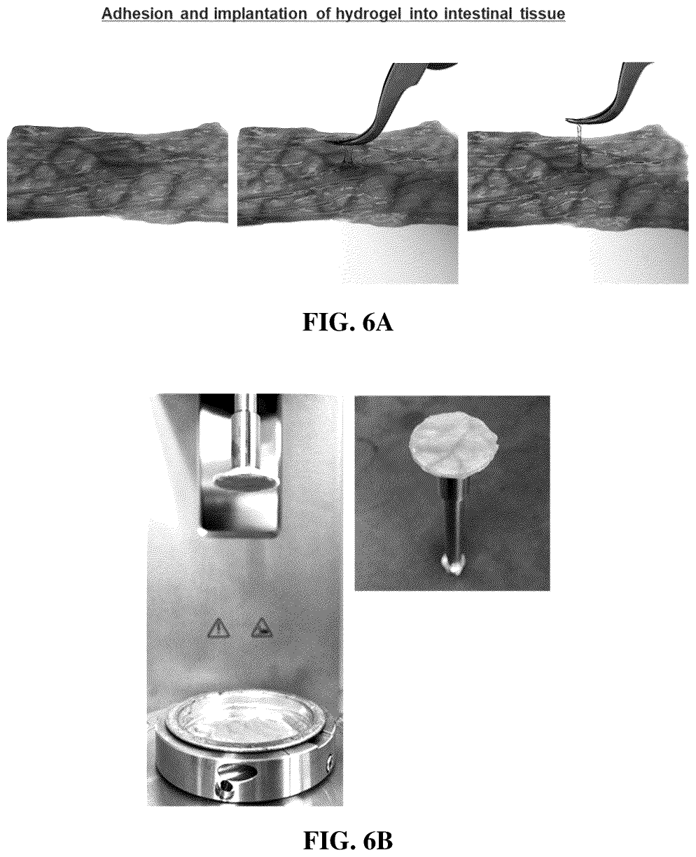

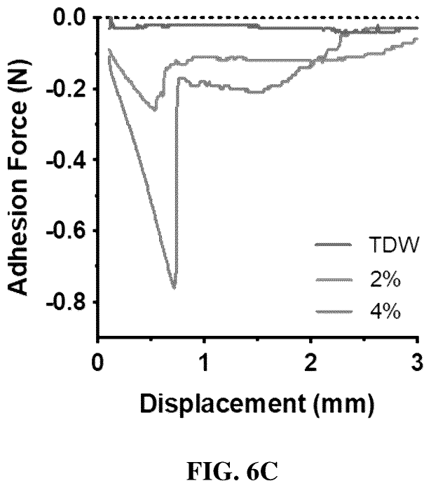

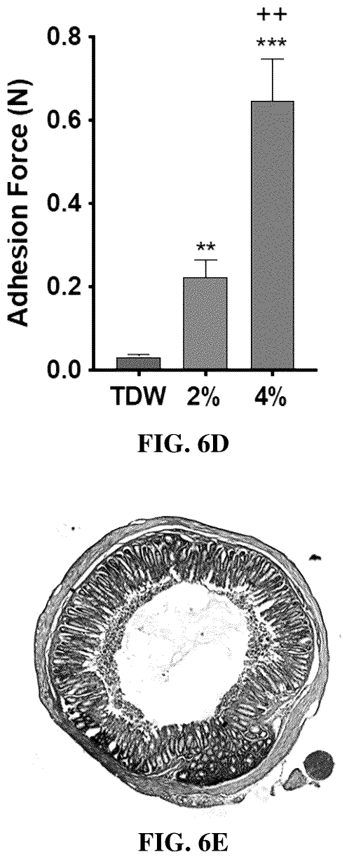

As a result, as seen in FIGS. 6A and 6B, it was confirmed that the hydrogel exhibited high adhesiveness to intestinal tissues and adhered well. In addition, as shown in FIGS. 6C and 6D, the results of rheological analysis showed that the hydrogel produced using the dopamine-modified carboxymethyl cellulose solution prepared at a concentration of 4 wt % had a high adhesion force to the tissue, suggesting that the hydrogel has a sufficient adhesion force to adhere to and remain in in vivo tissue.

In addition, 6 hours after the hydrogel produced using the dopamine-modified carboxymethyl cellulose solution prepared at a concentration of 4 wt % was injected into the mouse intestine, the hydrogel was visualized by H&E staining.

As a result, as shown in FIG. 6E, it was confirmed that the hydrogel had a sufficient adhesion force to adhere well to and remain on the surface of the intestinal tissue lumen.

### Experimental Example 1-6. Analysis of Internal Structure of Hydrogel

The internal structures of the hydrogels produced using different concentrations (2 wt % and 4 wt %) of dopamine-modified carboxymethyl cellulose according to the present invention were analyzed.

Specifically, the internal structures of the hydrogels were photographed using a field emission scanning electron microscope (FE-SEM).

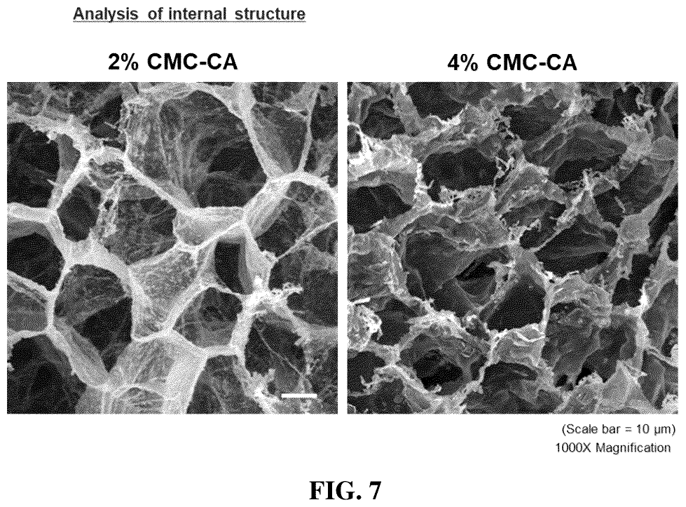

As a result, as shown in FIG. 7, it was confirmed that the hydrogels had a microporous structure, suggesting that they can be used for three-dimensional culture of cells and as a drug delivery platform.

### Experimental Example 1-7. Evaluation of Biocompatibility of Hydrogel

The biocompatibility of the dopamine-modified carboxymethyl cellulose hydrogel of the present invention was evaluated through three-dimensional cell culture.

Specifically, human adipose-derived stem cells (hADSCs) (1.0×10⁶ cells/100 μL of hydrogel) were loaded in the hydrogels produced using different concentrations (2 wt % and 4 wt %) of dopamine-modified carboxymethyl cellulose, and the cells were subjected to live/dead staining during three-dimensional culture. The live/dead staining was performed using the Live/Dead Viability/Cytotoxicity Kit (Invitrogen, Carlsbad, CA, USA) according to the manufacturer's instructions, and the cell viabilities on days 0 and 7 were measured. The stained cells were observed using a model IX73 fluorescence microscope (Olympus, Tokyo, Japan), and the ratio of viable cells (indicated in green) to dead cells (indicated in red) was quantified by manual counting from the images (n=3) obtained by the microscope.

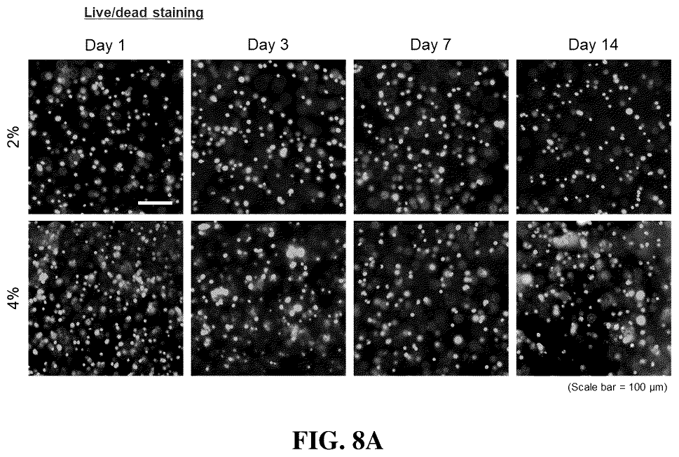

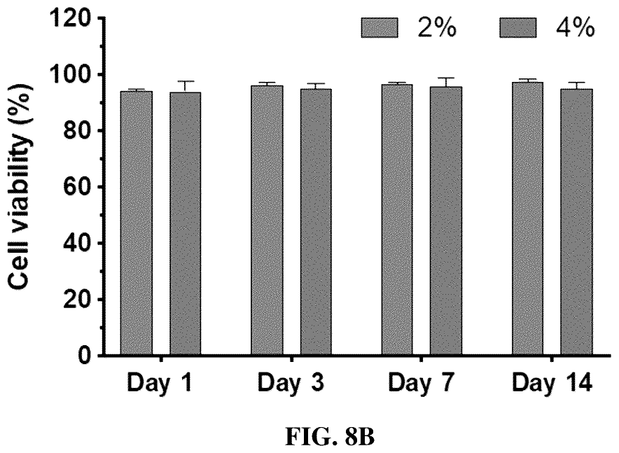

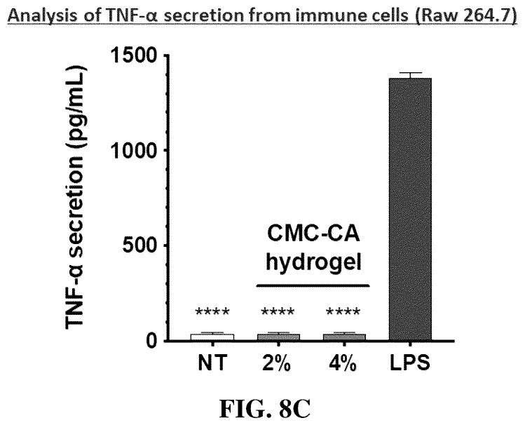

As a result, as shown in FIGS. 8A and 8B, it was confirmed that more than 90% of the cells cultured in the hydrogels produced using all of the concentrations survived, suggesting that the hydrogels have little cytotoxicity and excellent biocompatibility.

In addition, it was checked whether the dopamine-modified carboxymethyl cellulose hydrogel of the present invention would induce an inflammatory response.

Specifically, the dopamine-modified carboxymethyl cellulose hydrogel was incubated with macrophages (Raw 264.7), and the amount of tumor necrosis factor (TNF-α) secreted by the macrophages during inflammatory response was measured.

As shown in FIG. 8C, it was confirmed that the macrophages incubated with the dopamine-modified carboxymethyl cellulose hydrogel secreted TNF-α at a level similar to that of the control group without any treatment. These results confirm that the dopamine-modified carboxymethyl cellulose hydrogel is not likely to induce an inflammatory response even when applied in vivo.

### Experimental Example 1-8. Evaluation of In Vivo Toxicity of Hydrogel

The in vivo toxicity of the dopamine-modified carboxymethyl cellulose hydrogel of the present invention was evaluated.

Specifically, the dopamine-modified carboxymethyl cellulose hydrogel was implanted in mouse subcutaneous tissue, harvested together with the surrounding tissue, frozen with OCT, sectioned, and then analyzed by H&E tissue staining and toluidine blue staining.

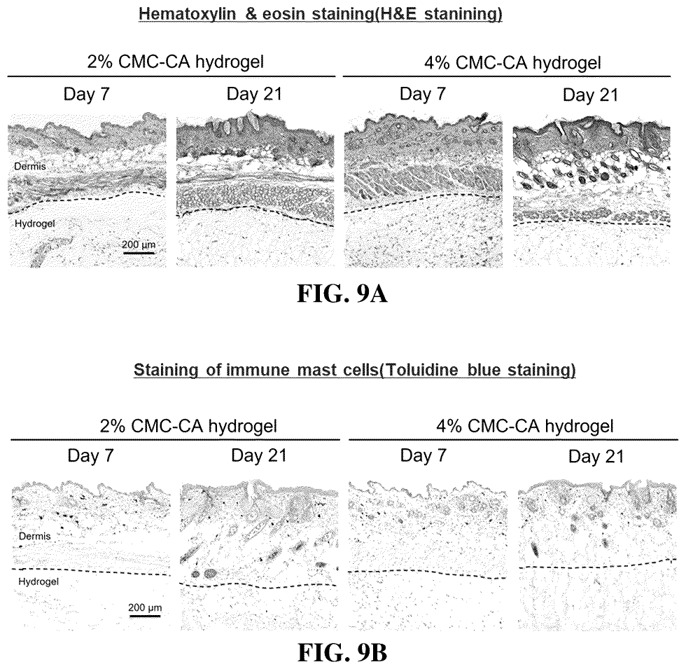

As a result, as shown in FIG. 9A, the results of H&E staining showed that no immune or inflammatory response occurred in the area implanted with the hydrogel. In addition, as shown in FIG. 9B, the results of toluidine blue staining showed that no mast cells were found in the implanted tissue area, indicating that no immune response occurred.

### Experimental Example 1-9. Evaluation of Hemostatic Performance of CMC-CA Hydrogel

To evaluate the hemostatic effect of the dopamine-modified carboxymethyl cellulose hydrogel in vivo, a liver bleeding model was created using 4-week-old female ICR mice (Orient Bio, Seongnam, Korea). Specifically, the mice were anesthetized, the abdomen was incised, sterilized filter paper was placed under the liver, bleeding was induced using an 18G needle, and the injured area was immediately treated with a 4 wt % dopamine-modified carboxymethyl cellulose hydrogel, a 4 wt % dopamine-modified carboxymethyl cellulose hydrogel patch, or a commercially available hemostatic agent (fibrin glue). Thereafter, the filter paper was replaced every 30 seconds until observation was completed, the bleeding profile was checked through the collected filter paper, and the amount of bleeding was measured by measuring the weight. After completing the evaluation of bleeding, the peritoneum and incision site were sutured with 6-0 Prolene suture. An untreated mouse was used as a control. After 7 days of treatment, the mice were sacrificed and their physiological status was examined.

From the mouse liver bleeding model, an untreated control group (No Treatment, NT), a group treated with a commercially available hemostatic agent (fibrin glue), a group treated with a 4 wt % dopamine-modified carboxymethyl cellulose hydrogel (hCMC-CA), and a group treated with a 4 wt % dopamine-modified carboxymethyl cellulose hydrogel patch (pCMC-CA) were was prepared, and the amount of bleeding in each group was then measured.

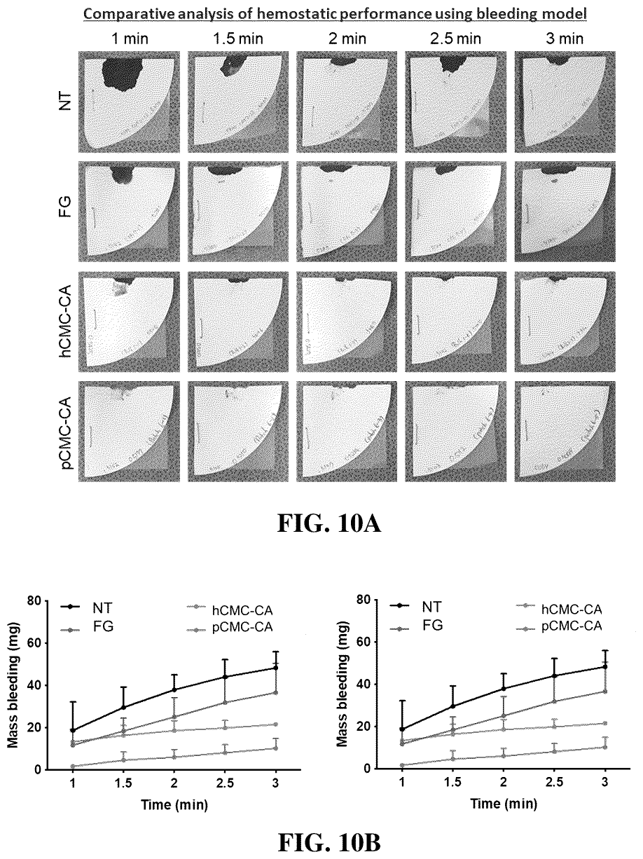

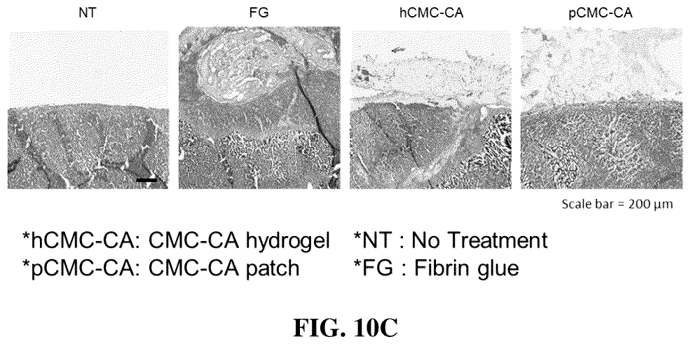

As a result, as shown in FIG. 10A, it was quantitatively confirmed that treatment with the dopamine-modified carboxymethyl cellulose hydrogel patch exhibited a significantly better hemostatic effect than treatment with the commercially available hemostatic agent or the dopamine-modified carboxymethyl cellulose hydrogel.

In addition, time-dependent photographs of the blood filter paper used to measure the amount of bleeding were checked. As a result, as shown in FIG. 10B, it was confirmed that bleeding stopped quickly when treated with the dopamine-modified carboxymethyl cellulose hydrogel patch or hydrogel.

As a result of performing H&E histological analysis of the treated area 3 days after hemostasis, it could be seen that the dopamine-modified carboxymethyl cellulose hydrogel patch effectively induced hemostatic action by inducing blood clot formation at the bleeding site (FIG. 10C).

### Experimental Example 1-10. Characterization of Hydrogel Patch

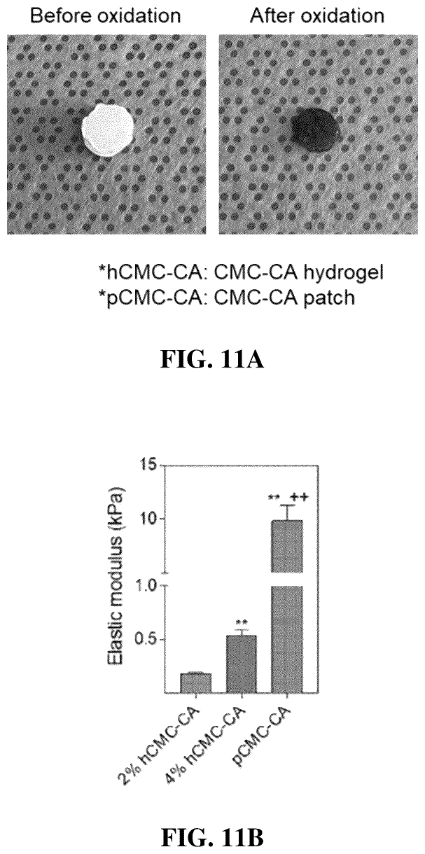

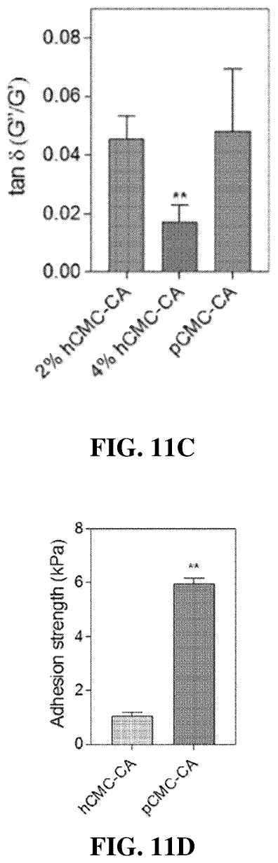

A CMC-CA patch was produced by rapidly cooling a CMC-CA hydrogel solution (2 wt %) in a mold having a desired size and shape, followed by freeze-drying. For analysis and application of the produced CMC-CA patch, crosslinking of the patch was induced by treatment with an oxidizing agent (4.5 mg/mL NaIO₄ solution) as a crosslinking agent (FIG. 11A).

It was confirmed that the CMC-CA patch had a significantly higher modulus than the 4 wt % CMC-CA hydrogel (4 wt % CMC-CA hydrogel=0.54 kPa at 1 Hz, CMC-CA patch=9.86 kPa at 1 Hz) (FIG. 11B).

It was confirmed that the average elasticity of the CMC-CA patch (0.048) was higher than that of the 4 wt % CMC-CA hydrogel (0.017), but was similar to that of the 2 wt % CMC-CA hydrogel (0.045). In other words, it could be seen that the CMC-CA patch had elasticity lower than that of the 4 wt % CMC-CA hydrogel, but had excellent elasticity similar to that of the 2 wt % CMC-CA hydrogel while having excellent mechanical properties (modulus) (FIG. 11).

The results of rheological analysis showed that the adhesion force of the CMC-CA patch to liver tissue (8 mm probe, 10μ/s detachment=5.97±0.20 kPa) was also higher than that of high-concentration (4% w/v) CMC-CA hydrogel (8 mm probe, 10μ/s detachment 4% hydrogel=1.06±0.12 kPa) (FIG. 11D).

In conclusion, the CMC-CA patch can be expected to be advantageous for practical use, because it has better physical properties and adhesion forces than the CMC-CA hydrogel formulation and is easy to store and use.

### Example 2: Synthesis of 5-Hydroxydopamine-Modified Carboxymethyl Cellulose Derivative and Hydrogel

### Example 2-1: Synthesis of 5-hydroxydopamine-Modified Carboxymethyl Cellulose Derivative

To synthesize a 5-hydroxydopamine-modified carboxymethyl cellulose derivative, carboxymethyl cellulose (CMC) was modified with 5-hydroxydopamine using EDC/NHS chemical reaction.

Specifically, carboxymethyl cellulose was dissolved in triple-distilled water (TDW) or 2-(N-morpholino) ethanesulfonic acid (MES) buffer solution at pH 5.5. 1-(3-dimethylaminopropyl)-3-ethylcarbodiimide hydrochloride (EDC, Thermo Fisher Scientific, Waltham, MA, USA) and N-hydroxysuccinimide (NHS, Sigma-Aldrich, St. Louis, MO, USA) were added to the solution, followed by stirring at pH 5.5 to 6.0 for 30 minutes.

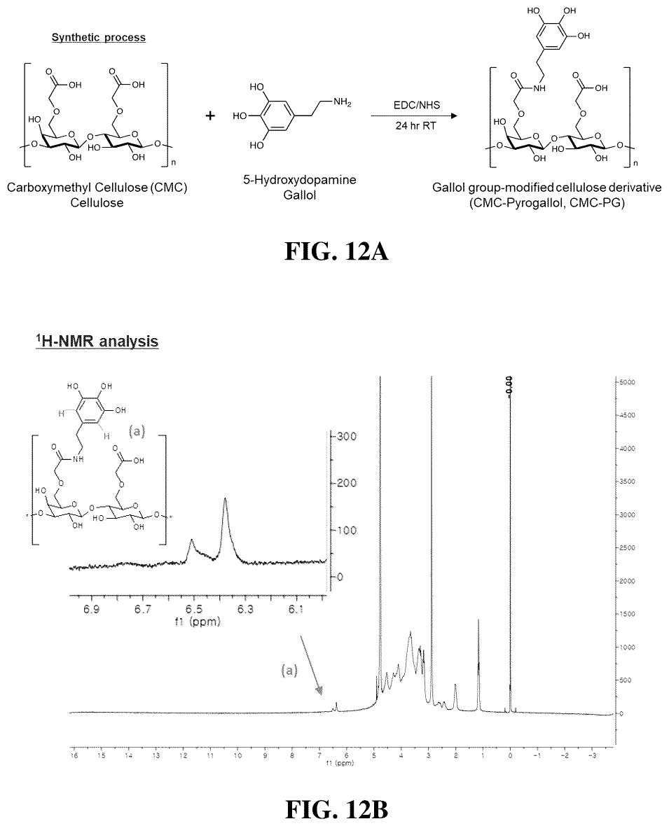

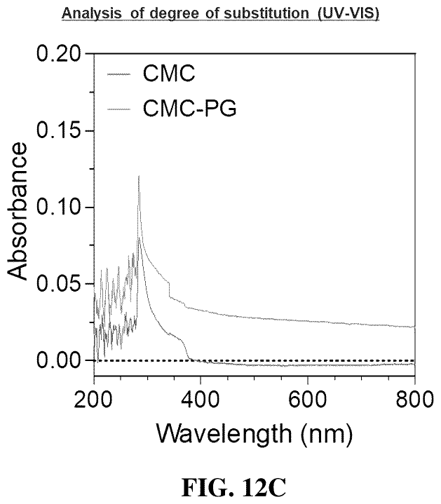

5-hydroxydopamine was added to the solution which was then stirred overnight at room temperature and pH 5.5 to 6.0, thereby synthesizing a 5-hydroxydopamine-modified carboxymethyl cellulose derivative. At this time, carboxymethyl cellulose (CMC):EDC:NHS:5-hydroxydopamine (CA) were mixed and reacted at a molar ratio of 1:2:2:2 (FIG. 12A).

The 5-hydroxydopamine-modified carboxymethyl cellulose derivative produced through the above-described process was analyzed by 1H-NMR (300 MHZ, Bruker, Billerica, MA, USA). As a result, as shown in FIG. 12B, it was confirmed that 5-hydroxydopamine was bound to carboxymethyl cellulose, and the peak values corresponding to 5-hydroxydopamine and carboxymethyl cellulose, respectively, appeared.

In addition, the degree of substitution with the gallol group for the CMC polymer was analyzed by UV-VIS spectrometry. As a result, it was confirmed that about 14% of the carboxyl groups were substituted with the gallol group (FIG. 12C).

### Example 2-2. Production of Hydrogel

A cellulose hydrogel was produced by crosslinking the 5-hydroxydopamine-modified carboxymethyl cellulose derivative of Example 2-1. Specifically, a hydrogel was produced by inducing crosslinking by an oxidation reaction with sodium periodate (NaIO₄) or sodium hydroxide (NaOH).

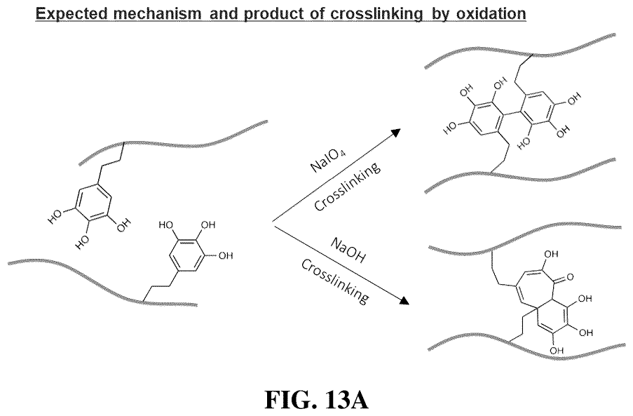

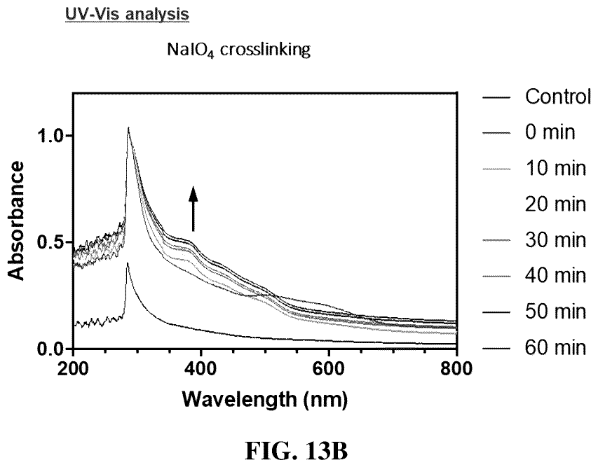

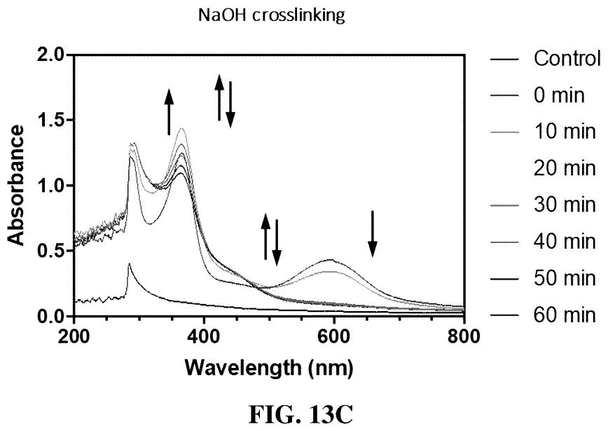

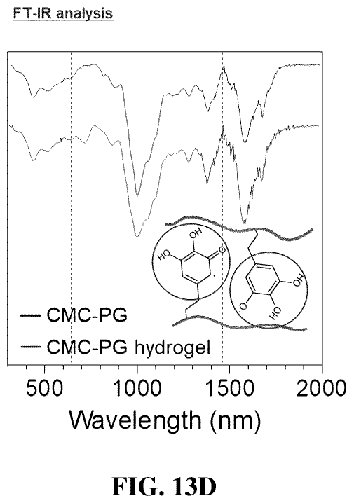

As shown in FIG. 13A, it was confirmed that crosslinking of the carboxymethyl cellulose derivative was achieved by chemical bonding between the oxidized gallol groups, and the obtained hydrogel was slightly yellow.

In order to specifically confirm the crosslinking of the carboxymethyl cellulose hydrogel, the spectra before and after oxidation were compared through ultraviolet-visible (UV-Vis) spectroscopy.

As a result, an absorbance peak at 280 to 300 nm was observed through UV-Vis analysis, indicating the presence of oxides containing crosslinked gallol groups. Specifically, when oxidized with sodium periodate, it was observed that peaks corresponding to the semi-quinone intermediate and phenoxyl radical identified during the oxidation of the gallol groups appeared in the range of 350 to 380 nm (FIG. 13B).

In addition, when oxidized with sodium hydroxide, a peak corresponding to purpurogallin was observed in the range of 420 to 440 nm. It was confirmed that a broad peak was observed at around 600 nm, indicating that a charge transfer complex was formed by intermolecular interactions (FIG. 13C).

The results of FT-IR analysis showed that, after treatment with the oxidizing agent (NaIO₄), the crosslinking reaction of the CMC-PG derivative proceeded through the formation of phenoxyl radicals (\textcircled{1} ˜700 mm), which are an oxidized gallol derivative, and the formation of biphenolic ester between the phenoxyl radicals (\textcircled{2} ˜1,400 nm) (FIG. 13D).

### Experimental Example 2: Characterization of 5-Hydroxydopamine-Modified Carboxymethyl Cellulose

### Experimental Example 2-1. Examination of Changes in Physical Properties Depending on Crosslinking Conditions and Hydrogel Derivative Concentration

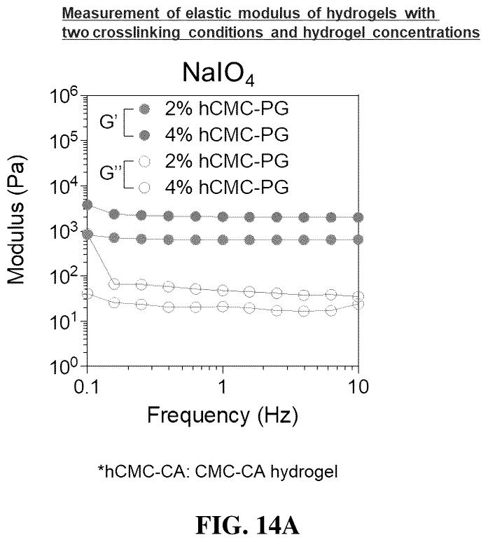

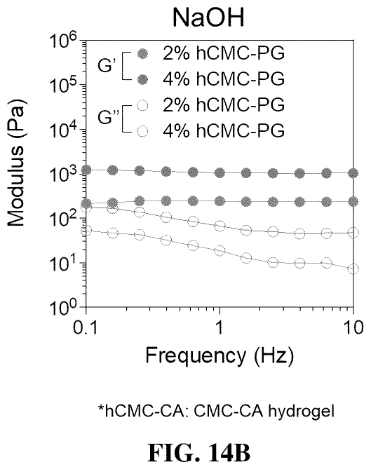

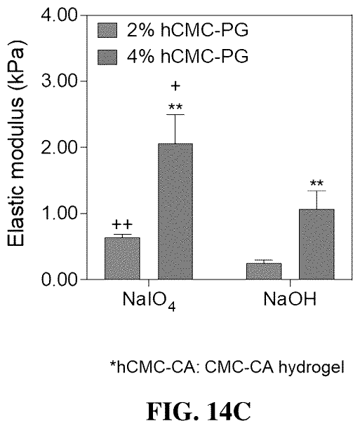

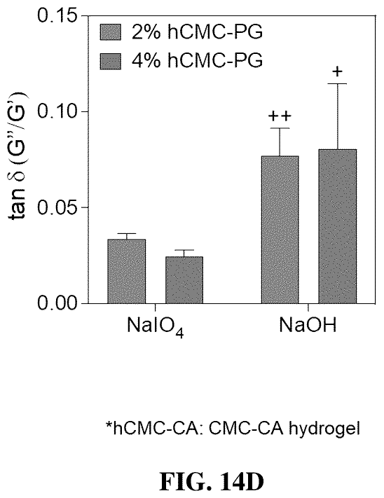

The differences in physical properties of cellulose hydrogels formed using CMC-PG solutions prepared at two concentrations (2 wt % and 4 wt %) were examined through rheological analysis. It was confirmed that, as the concentration of the CMC-PG solution increased, the elastic modulus, which represents the mechanical properties of the hydrogel, increased, but the tan δ value, which represents the elasticity of the hydrogel, did not significantly change. (FIGS. 14A to 14D).

The differences in physical properties of cellulose hydrogels formed according to the two oxidation methods were examined through rheological analysis (FIGS. 14A to 14C). As a result, it was confirmed that, under each concentration condition, the elastic modulus was higher in the crosslinking method performed using NaIO₄ (2%=0.63±0.05 kPa, 4%=2.06±0.43 kPa) than in the crosslinking method performed using NaOH (2%=0.25±0.05 kPa, 4%=1.07±0.27 kPa), and the tan δ value was also higher in the crosslinking method performed using NaIO₄ than in the crosslinking method performed using NaOH (NaIO₄: 2%=0.03±0.01, 4%=0.02±0.003, NaOH: 2%=0.08±0.01, 4%=0.08±0.03).

That is, it was confirmed that, when the oxidation method using NaIO₄ was applied, it was possible to produce CMC-PG hydrogels having better physical properties and elasticity.

### Experimental Example 2-2. Analysis of Swelling Profiles and Internal Structures of Hydrogels

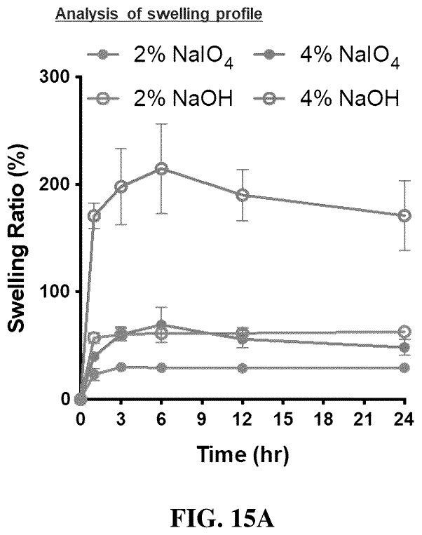

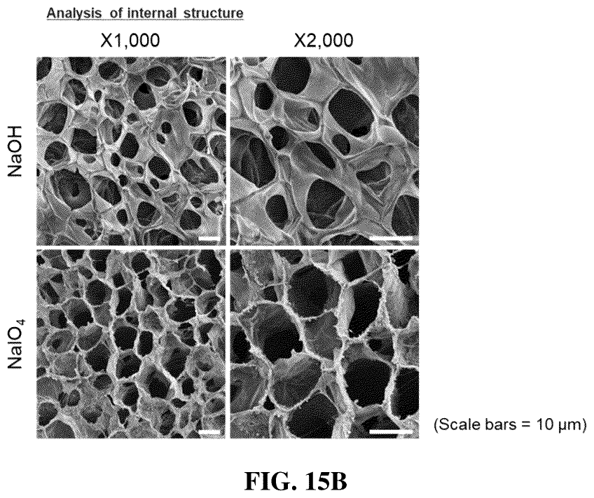

Cellulose hydrogels were formed using two crosslinking methods (NaIO₄ and NaOH) under two CMC-PG concentration conditions (2 wt % and 4 wt %), and then the swelling profiles of the hydrogels were measured. As a result, as shown in FIG. 15A, it was confirmed that the hydrogel obtained by crosslinking by NaOH treatment swelled more than the hydrogel obtained by crosslinking by NaIO₄ treatment. In addition, it was confirmed that the hydrogel swelled more under the 4 wt % condition than under the 2 wt % condition, under both the two crosslinking conditions. Thus, it can be seen that the swelling profile can be adjusted depending on the concentration condition or crosslinking condition of CMC-PG used.

Using a field-emission scanning electron microscopy (FE-SEM) system, it was confirmed that the internal structures of the CMC-PG hydrogels obtained by crosslinking using the two methods had a microporous structure (FIG. 15B).

These results confirm that the CMC-PG hydrogel can also be used for three-dimensional culture of cells or as a drug delivery platform.

### Experimental Example 2-3. Analysis of Adhesive Properties of Hydrogels

To evaluate the adhesive properties of the hydrogels produced in the present invention, rheological analysis was performed.

Specifically, the adhesion forces of the hydrogels formed using different CMC-PG concentrations and an oxidizing agent (NaIO₄) were measured using a rheological analysis device.

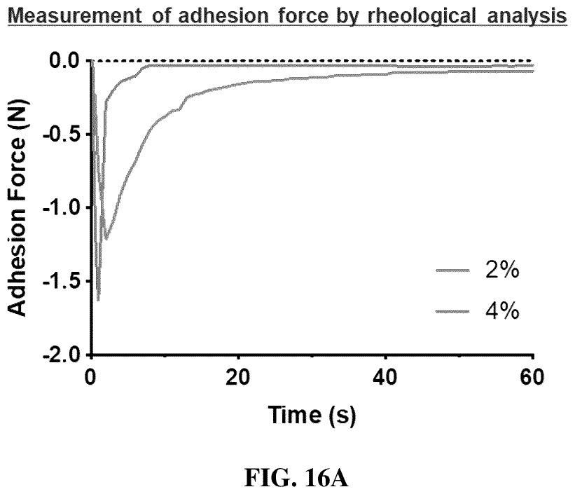

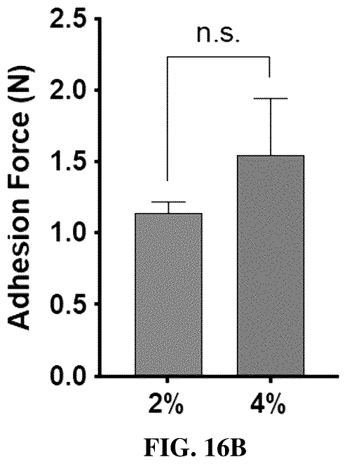

The results of rheological analysis showed that the adhesion force of the 4 wt % CMC-PG hydrogel with a high concentration was higher than that of the 2 wt % CMC-PG hydrogel (FIGS. 16A and 16B).

This suggests that it is possible to adjust the adhesion force depending on the concentration of the CMC-PG derivative.

### Experimental Example 2-4. Evaluation of pH Sensitivity of Hydrogels

The pH sensitivity of the hydrogels produced in the present invention was evaluated by measuring the pH-dependent change in the size of the hydrogel.

Specifically, the hydrogels produced by preparing 5-hydroxydopamine-modified carboxymethyl cellulose (CMC-PG) solutions at concentrations of 2 wt % and 4 wt % and treating the solutions with sodium periodate were immersed in solutions with different pHs (pH 1, pH 7.4, and pH 14), and changes in the volume thereof were checked.

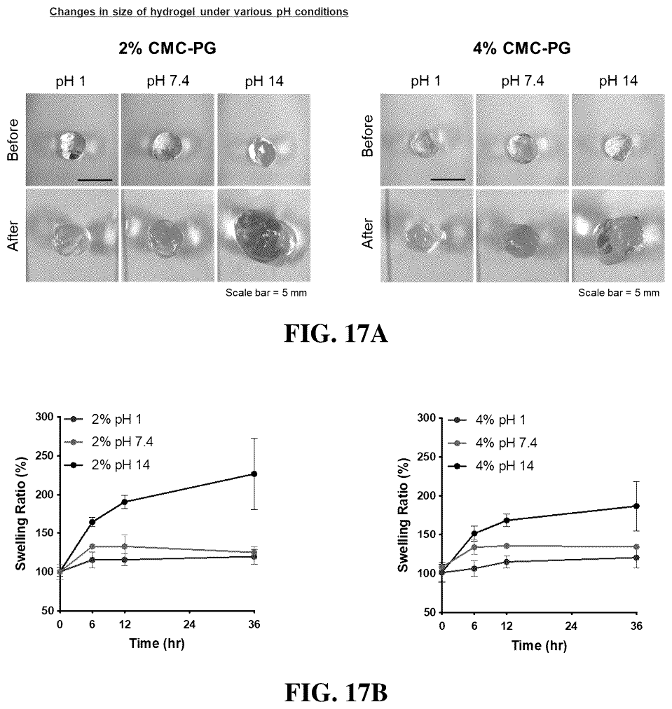

As a result, as shown in FIG. 17, it was confirmed that the size and volume of the hydrogel were maintained in the acidic low-pH solution, but the volume of the hydrogel slightly increased in the neutral pH solution, and the volume of the hydrogel significantly increased in the alkaline pH solution.

These results suggest that the volume (size) of the hydrogel can change depending on pH conditions, indicating that the hydrogel is pH-sensitive.

In addition, the produced hydrogel was observed using a field-emission scanning microscope (FE-SEM) system.

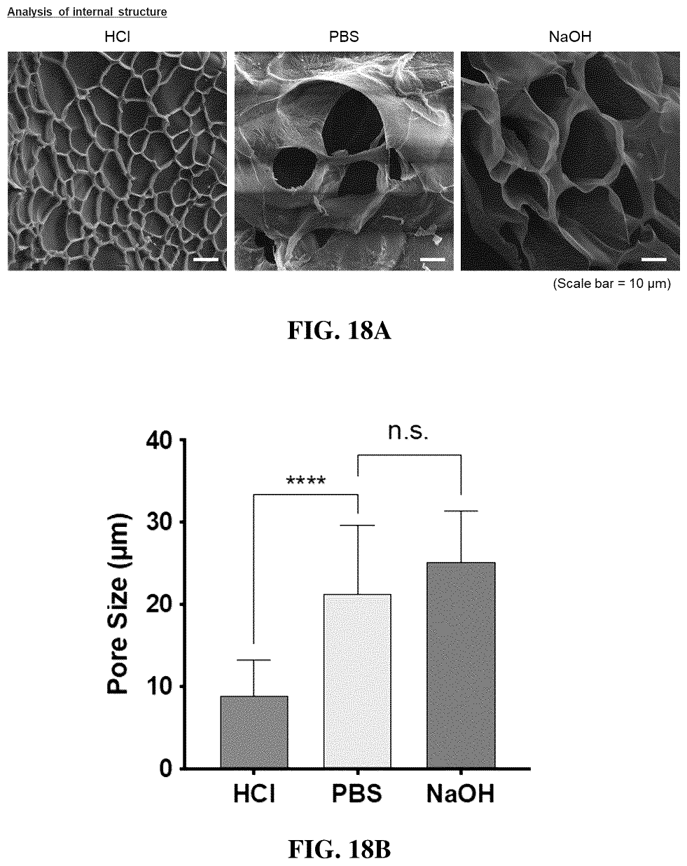

As a result, as shown in FIG. 18, it could be confirmed that the pore size of the hydrogel changed depending on pH. Specifically, it could be confirmed that, at an acidic low pH, the hydrogel showed the smallest porous structure, and as the pH increased, the pore size increased.

From these results, it is believed that the pH-dependent change in the size of the hydrogel is due to the change in the size of the internal pores. This characteristic suggests that the hydrogel can be used as a pH-sensitive drug delivery system from which drug release is controlled depending on pH.

### Experimental Example 2-5. Evaluation of Applicability as pH-Sensitive Drug Delivery System

The applicability of the hydrogel of the present invention as the pH-sensitive drug delivery system described in Experimental Example 2-4 above was evaluated.

Specifically, the hydrogel was loaded with bovine serum albumin (BSA), and then placed in an acidic solution of pH 2, which was then replaced with a neutral solution of pH 7, and the release profile of BSA from the hydrogel was checked.

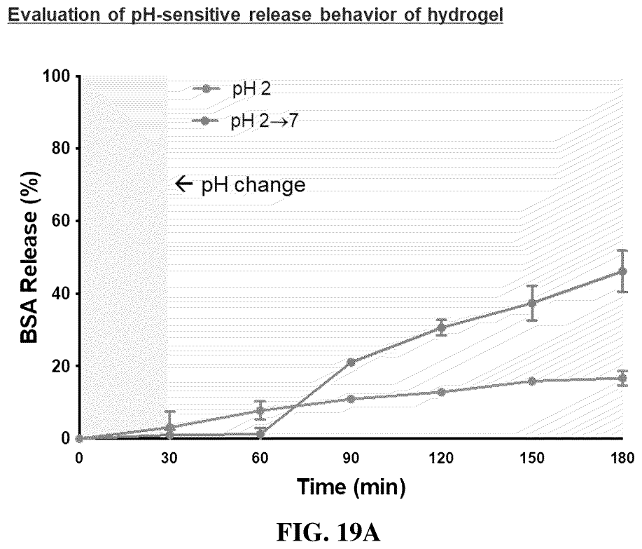

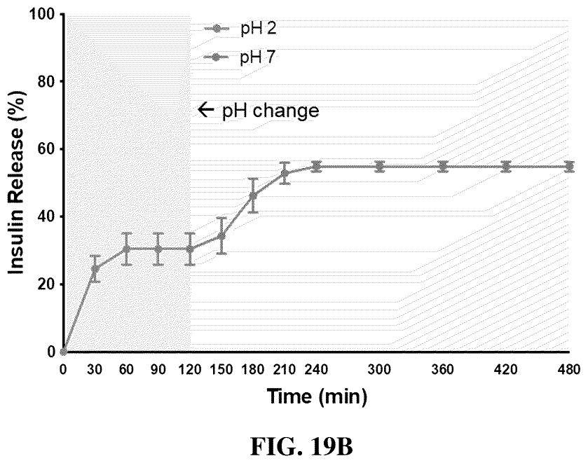

As a result, as shown in FIG. 19A, it was confirmed that BSA was released after the hydrogel was placed at pH 7. This suggests that, under low-pH conditions, the pore size of the hydrogel is reduced and BSA release therefrom does not occur, but under high-pH conditions, the hydrogel swells and BSA is released therefrom.

In addition, the hydrogel was loaded with insulin instead of BSA, and the profile of insulin release therefrom was evaluated by conducting the experiment under the same conditions.

As a result, as shown in FIG. 19B, it was confirmed that only a small amount of insulin was released under low-pH conditions, but a larger amount of insulin was released in a high-pH solution.

These results suggest that the hydrogel of the present invention can be loaded with a drug, can retain the loaded drug without damage under low-pH conditions, and can release the drug under high-pH conditions so that the drug can be effectively released and delivered to a desired location. This indicates the possibility of a mechanism in which the hydrogel is loaded with an oral drug, and does not release the drug in the stomach (low pH), but releases the active ingredient in the intestines (high pH).

### Experimental Example 2-6. Evaluation of Biocompatibility (Evaluation of Toxicity) of Hydrogel

The biocompatibility of the 5-hydroxydopamine-modified carboxymethyl cellulose hydrogel of the present invention was evaluated through three-dimensional cell culture.

Specifically, human adipose-derived stem cells (hADSCs) (1.0×10⁶ cells/100 μL of hydrogel) were loaded in the hydrogels produced using different concentrations (2 wt % and 4 wt %) of 5-hydroxydopamine-modified carboxymethyl cellulose and each oxidizing agent (sodium periodate or sodium hydroxide), and the cells were subjected to live/dead staining during three-dimensional culture. The live/dead staining was performed using the Live/Dead Viability/Cytotoxicity Kit (Invitrogen, Carlsbad, CA, USA) according to the manufacturer's instructions, and the cell viabilities on days 0 and 7 were measured. The stained cells were observed using a model IX73 fluorescence microscope (Olympus, Tokyo, Japan), and the ratio of viable cells (indicated in green) to dead cells (indicated in red) was quantified by manual counting from the images (n=3) obtained by the microscope.

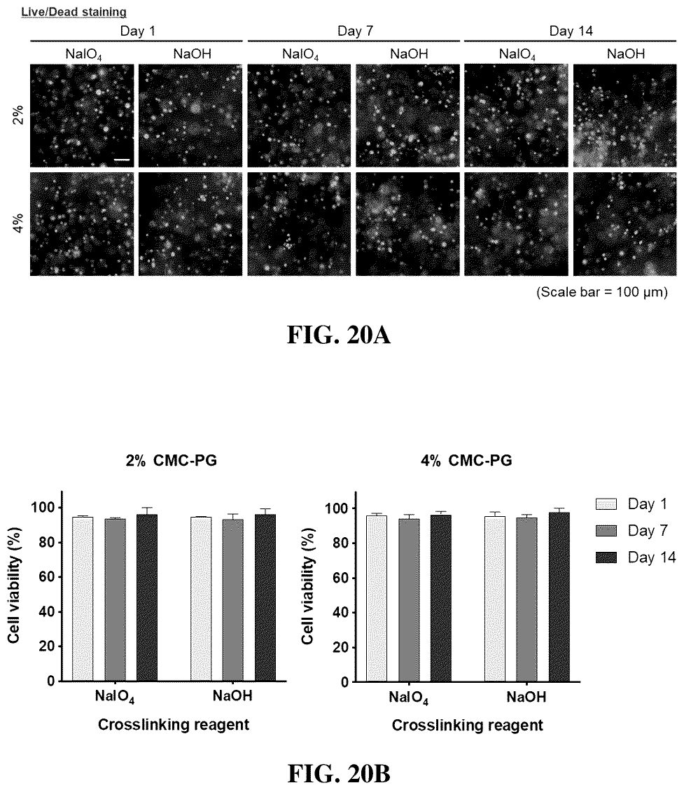

As a result, as shown in FIG. 20, it was confirmed that more than 90% of the cells cultured in the hydrogels produced using all of the concentrations and each oxidizing agent survived, suggesting that the hydrogels have little cytotoxicity and excellent biocompatibility.

### Experimental Example 2-7. Evaluation of Applicability of Hydrogel as Filler

It was evaluated whether the hydrogel of the present invention would be applied as a filler material.

Specifically, the applicability of the hydrogel as a filler was evaluated by administering 4 wt % of 5-hydroxydopamine-modified carboxymethyl cellulose (CMC-PG) without crosslinking to mouse subcutaneous tissue.

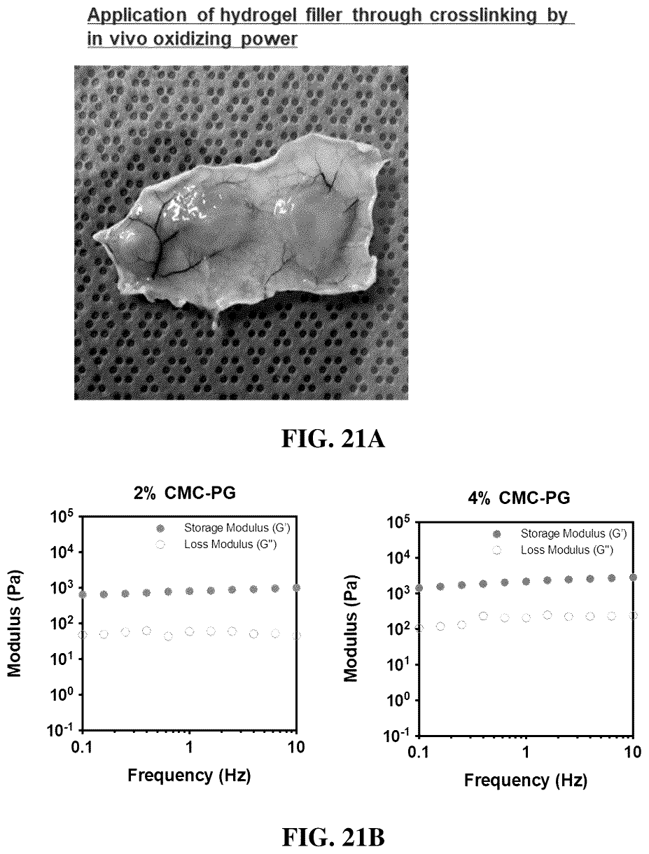

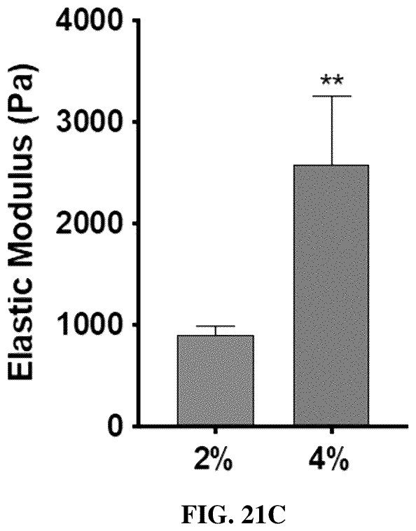

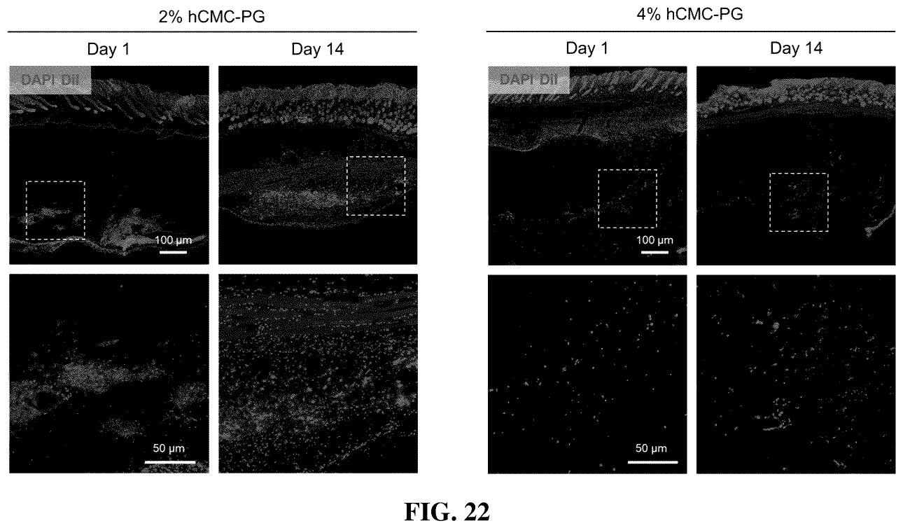

As a result, as shown in FIG. 21A, it was confirmed that a hydrogel was formed in vivo even without a separate oxidizing agent due to the oxidizing ability of 5-hydroxydopamine.

Next, the formed hydrogel was collected and the physical properties thereof were measured in the same manner as Experimental Example 2-1.

As a result, as shown in FIGS. 21B and 21C, it was confirmed that the physical properties of the hydrogel formed in vivo also changed depending on the concentration of 5-hydroxydopamine-modified carboxymethyl cellulose, suggesting that the physical properties are adjustable.

Taking these results together, it can be seen that, since the hydrogel of the present invention is based on cellulose that has excellent physical properties and exists stably in vivo, it is a material suitable as a filler that is used for cosmetic and plastic surgery purposes where maintaining the volume thereof in vivo for a long period of time is important.

### Experimental Example 2-8. Evaluation of Applicability of Hydrogel as Material for Cell Transplantation

It was evaluated whether the hydrogel of the present invention would be used as a material for cell transplantation.

Specifically, human adipose-derived stem cells (hADSCs) labeled with a fluorescent dye (Dil) and non-crosslinked 5-hydroxydopamine-modified carboxymethyl cellulose (hCMC-PG) were mixed together and injected into mouse subcutaneous tissue without a crosslinking agent.

As a result, as shown in FIG. 22, it was confirmed that a hydrogel was formed in vivo even without a separate oxidizing agent due to the oxidizing ability of 5-hydroxydopamine, and that the transplanted cells were maintained within the hydrogel for 2 weeks.

Taking these results together, it can be seen that the hydrogel of the present invention is applicable as a material for cell transplantation.

### Experimental Example 2-9. Production and Characterization of CMC-PG Patch

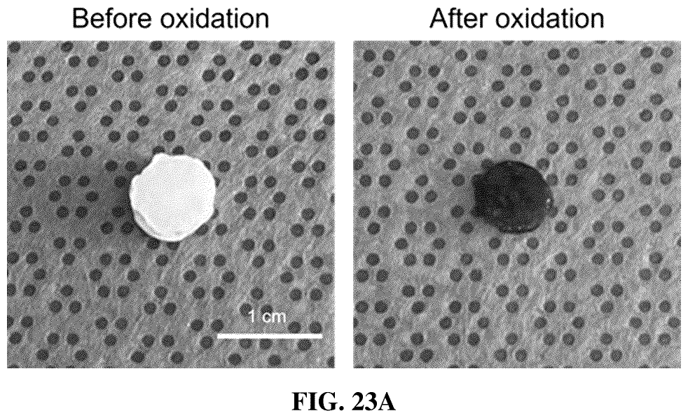

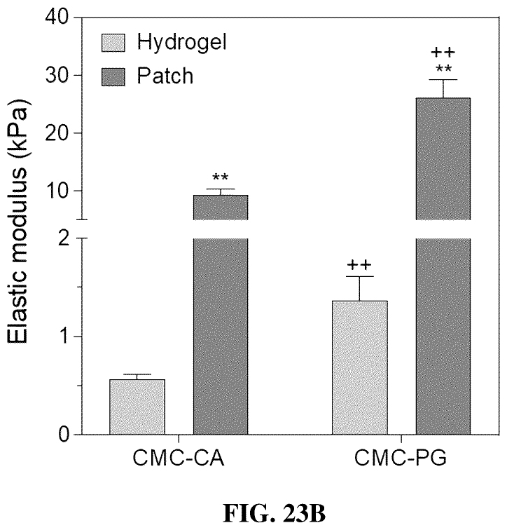

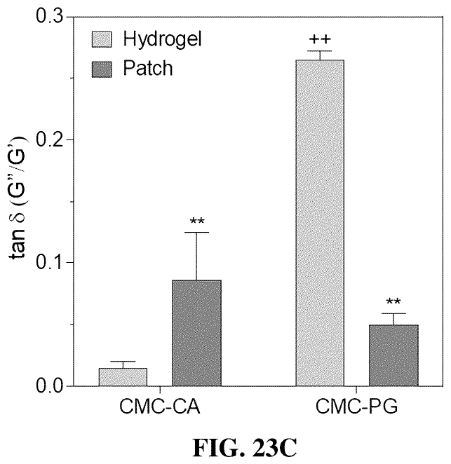

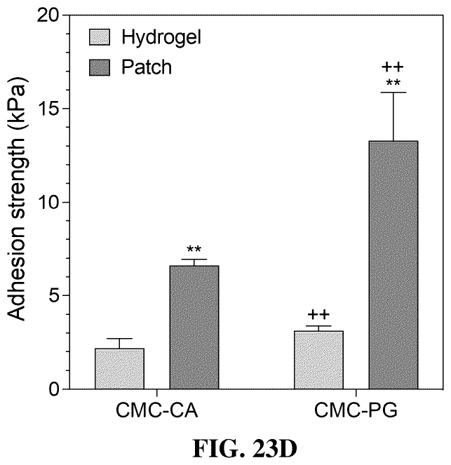

A CMC-PG patch was produced by rapidly cooling a CMC-CA hydrogel solution (2 wt %) in a mold having a desired size and shape, followed by freeze-drying. The CMC-PG patch has an advantage in that the CMC-PG patch, when applied in vivo, is self-crosslinkable through natural oxidation by oxygen in the tissue due to the high oxidation power of the gallol group (PG), and thus is capable of forming a hydrogel in vivo even without additional treatment with a crosslinking agent. In an ex vivo environment, for ease of analysis, the CMC-PG patch was crosslinked by treatment with an oxidizing agent (4.5 mg/mL NaIO₄ solution) and then analyzed (FIG. 23A).

The results of rheological analysis showed that the modulus of the CMC-PG patch (26.20±3.01 kPa at 1 Hz) was higher than that of the CMC-PG hydrogel (1.37±0.24 kPa) and that of the CMC-CA-based hydrogel or patch (CMC-CA hydrogel=0.56±0.05 kPa, CMC-CA patch=9.44±0.96 kPa) (FIG. 23B).

The tan δ value of the CMC-PG patch (0.05) was slightly lower than the tan δ value of the CMC-CA patch (0.086) and significantly lower than that of the CMC-PG hydrogel (0.26), which means that the CMC-PG patch has excellent mechanical properties (modulus) while having excellent elasticity (FIG. 23C).

In addition, the results of rheological analysis that the adhesion force of the CMC-PG patch to porcine liver tissue (8 mm probe, 10μ/s detachment=13.28±2.59 kPa) was higher than that of the CMC-PG hydrogel (3.13±0.25 kPa) or the CMC-CA-based hydrogel or patch (CMC-CA hydrogel=2.19±0.53 kPa, CMC-CA patch=6.61±0.34 kPa) (FIG. 23D).

In conclusion, it can be confirmed that the CMC-PG patch is excellent in terms of both mechanical properties and tissue adhesion force compared to the CMC-CA-based material and the CMC-PG hydrogel formulations, suggesting that it is more suitable for tissue engineering applications.

### Experimental Example 2-10. Evaluation of Hemostatic Performance of CMC-PG Hydrogel and Patch

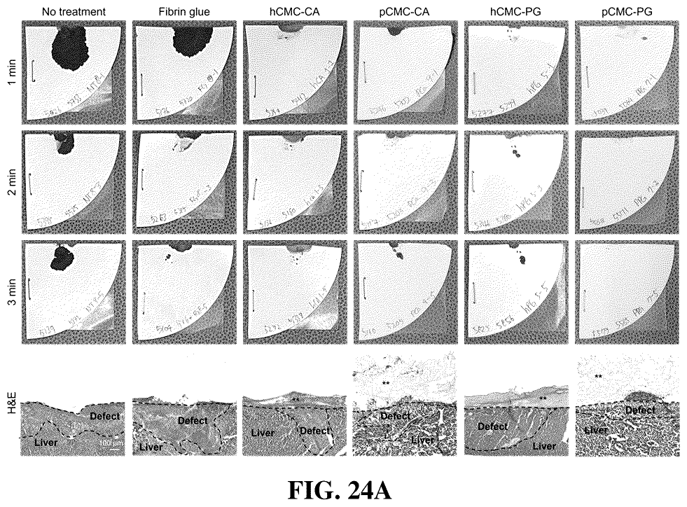

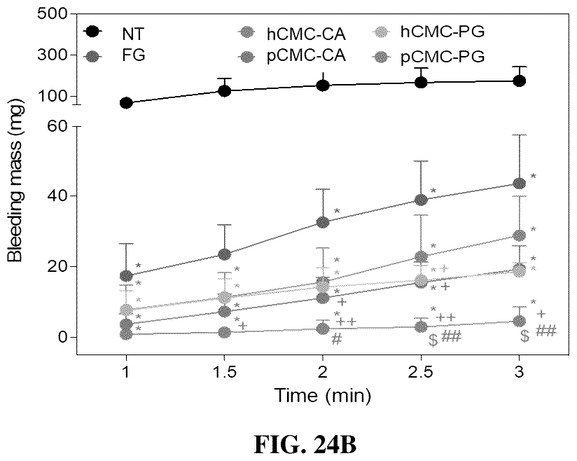

From a mouse liver bleeding model, an untreated control group (No Treatment, NT), a group treated with a commercially available hemostatic agent (fibrin glue; FG), groups treated with CMC-CA and CMC-PG hydrogels (2 wt %, hCMC-CA, and hCMC-PG), and groups treated with CMC-CA and CMC-PG patches (pCMC-CA, and pCMC-PG) were prepared, and the amount of bleeding in each group was then measured. It was confirmed that, when the CMC-PG patch with the best tissue adhesion force and physical properties was used, it exhibited better hemostatic performance than the same amount of the commercially available fibrin hemostatic agent or the CMC-PG and CMC-CA hydrogel formulations (FIGS. 24A and 24B).

Referring to time-dependent photographs of the blood-absorbing filter paper used to measure the amount of bleeding in the hemostatic performance evaluation experiment, it can be confirmed that the CMC-PG patch group exhibited excellent hemostatic ability from the beginning of bleeding compared to the other groups (FIG. 24A). The results of comparing the total amount of bleeding up to 3 minutes after bleeding indicated that the CMC-PG patch exhibited the best hemostatic performance (FIG. 24B).

The results of H&E histological analysis 3 days after treatment of the bleeding site confirmed that, among the patch groups, the CMC-PG patch, in particular, adhered stably and well to the tissue, forming a physical barrier which prevents additional bleeding (the bottom panel of FIG. 24A).

So far, the present invention has been described with reference to the embodiments. Those of ordinary skill in the art to which the present invention pertains will appreciate that the present invention may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

1. A hydrogel comprising cellulose modified with a phenol derivative.

2. The hydrogel of claim 1, wherein the phenol derivative is dopamine 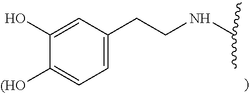
or 5-hydroxydopamine 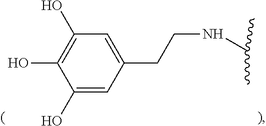 [SMILES: *NCCc1cc(O)c(O)c(O)c1]
and
the cellulose is any one or more of carboxymethyl cellulose, hydroxypropyl methylcellulose, cellulose acetate phthalate, hydroxypropyl methylcellulose acetate/succinate, hydroxyethyl cellulose, ethyl methyl cellulose, hydroxypropyl cellulose, cellulose propionate, and cellulose acetate butyrate.

3. The hydrogel of claim 1, wherein the hydrogel comprises a structure represented by Formula 1 below: 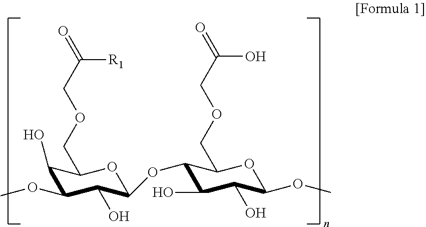
wherein
R₁ is

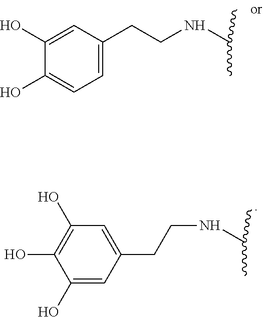 [SMILES: *NCCc1ccc(O)c(O)c1 ; *NCCc1cc(O)c(O)c(O)c1]

4. The hydrogel of claim 1, wherein the phenol derivative and the cellulose are at a molar ratio of 1:3 to 3:1.

5. The hydrogel of claim 1, wherein the hydrogel is used for one or more purposes selected from the group consisting of blood clotting promotion, hemostasis, cell differentiation promotion, cell culture, cell transplantation, and drug delivery.

6. The hydrogel of claim 1, wherein the hydrogel is adhesive.

7. The hydrogel of claim 1, wherein the hydrogel is biodegradable.

8. A hemostatic agent composition comprising the hydrogel of claim 1.

9. The hemostatic agent composition of claim 8, wherein the hemostatic agent is in the form of an adhesive patch or film.

10. A tissue adhesive composition comprising the hydrogel of claim 1.

11. A composition for cell culture and transplantation comprising the hydrogel of claim 1.

12. A composition for drug delivery comprising the hydrogel of claim 1.

13. The composition of claim 12, wherein the drug is loaded in the hydrogel.

14. The composition of claim 12, wherein the drug comprises one selected from the group consisting of immune cell activators, anticancer agents, therapeutic antibodies, antibiotics, antibacterial agents, antiviral agents, anti-inflammatory agents, contrast agents, protein drugs, growth factors, cytokines, peptide drugs, hair growth agents, anesthetics, and combinations thereof.

15. A method for producing a hydrogel comprising cellulose modified with a phenol derivative, the method comprising steps of:
a) substituting cellulose with the phenol derivative; and
b) crosslinking the substituted cellulose to form a hydrogel.

16. The method of claim 15, wherein the step of substituting cellulose with the phenol derivative comprises substituting a hydroxyl (—OH) group of cellulose with R₁ to produce a compound represented by Formula 1 below: 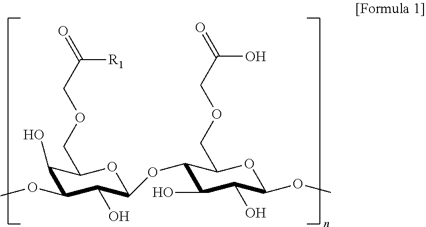
wherein
R₁ is

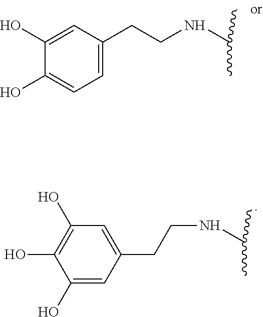 [SMILES: *NCCc1ccc(O)c(O)c1 ; *NCCc1cc(O)c(O)c(O)c1]

17. The method of claim 15, wherein step b) of crosslinking the substituted cellulose to form the hydrogel comprises treating the substituted cellulose with any one or more of NaOH, NaIO₄, Na₂S₂O₈, Fe³⁺, HNO₃, MnO₄²⁻, and H₂SO₄.
